SEQUENCE LISTING (106 sequences)

SEQ ID NO: 1 - protein, 5 aa, unidentified
NYLMN

SEQ ID NO: 2 - protein, 17 aa, unidentified
RIDPYDGDIDYNQNFKD

SEQ ID NO: 3 - protein, 10 aa, unidentified
GYGTAYGVDY

SEQ ID NO: 4 - protein, 15 aa, unidentified
RASESVDNYGISFMN

SEQ ID NO: 5 - protein, 8 aa, unidentified
AASRQGSG

SEQ ID NO: 6 - protein, 9 aa, unidentified
QQSKEVPWT

SEQ ID NO: 7 - protein, 129 aa, unidentified
EVQLVESGGGLVQPGGSLRLSCAASGFTFSNYLMNWVRQAPGKGLEWVRIDPYDGDIDYNQNFKDGGSTYYADSVKGRFTISADTSKNTAYLQMNSLRAEDTAVYYCAGYGTAYGVDYYWGQGTLVTVS

SEQ ID NO: 8 - protein, 118 aa, unidentified
DIQMTQSPSSLSASVGDRVTITCRASQRASESVDNYGISFMNAWYQQKPGKAPKAASRQGSGSGVPSRFSGSGSGTDFTLTISSLQPEDFATYYCQQSKEVPWTGTKVEIKRTVAAPS

SEQ ID NO: 9 - protein, 5 aa, unidentified
DSWIH

SEQ ID NO: 10 - protein, 6 aa, unidentified
AWISPY

SEQ ID NO: 11 - protein, 9 aa, unidentified
RRHWPGGFD

SEQ ID NO: 12 - protein, 6 aa, unidentified
DVSTAV

SEQ ID NO: 13 - protein, 10 aa, unidentified
LLIYSASFLY

SEQ ID NO: 14 - protein, 12 aa, unidentified
QQYLYHPATFGQ

SEQ ID NO: 15 - protein, 117 aa, unidentified
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVS

SEQ ID NO: 16 - protein, 114 aa, unidentified
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPS

SEQ ID NO: 17 - protein, 15 aa, unidentified
GGGGSGGGGSGGGGS

SEQ ID NO: 18 - protein, 523 aa, unidentified
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSGGGGSGGGGSGGGGSDIQMTQSPSSLSASVGDRVTITCRASQRASESVDNYGISFMNAWYQQKPGKAPKAASRQGSGSGVPSRFSGSGSGTDFTLTISSLQPEDFATYYCQQSKEVPWTGTKVEIKRTVAAPSGGGGSGGGGSGGGGSEVQLVESGGGLVQPGGSLRLSCAASGFTFSNYLMNWVRQAPGKGLEWVRIDPYDGDIDYNQNFKDGGSTYYADSVKGRFTISADTSKNTAYLQMNSLRAEDTAVYYCAGYGTAYGVDYYWGQGTLVTVS

SEQ ID NO: 19 - protein, 83 aa, unidentified
RSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 20 - protein, 112 aa, unidentified
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 66 aa, unidentified
TTTPAPRPPTPAPTIASQPLSLRPEACRPAAGGAVHTRGLDFACDIYIWAPLAGTCGVLLLSLVIT

SEQ ID NO: 22 - protein, 261 aa, unidentified
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 23 - protein, 246 aa, unidentified
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPS

SEQ ID NO: 24 - protein, 246 aa, unidentified
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVS

SEQ ID NO: 25 - protein, 523 aa, unidentified
DIQMTQSPSSLSASVGDRVTITCRASQRASESVDNYGISFMNAWYQQKPGKAPKAASRQGSGSGVPSRFSGSGSGTDFTLTISSLQPEDFATYYCQQSKEVPWTGTKVEIKRTVAAPSGGGGSGGGGSGGGGSEVQLVESGGGLVQPGGSLRLSCAASGFTFSNYLMNWVRQAPGKGLEWVRIDPYDGDIDYNQNFKDGGSTYYADSVKGRFTISADTSKNTAYLQMNSLRAEDTAVYYCAGYGTAYGVDYYWGQGTLVTVSGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPS

SEQ ID NO: 26 - protein, 66 aa, unidentified
IEVMYPPPYLDNEKSNGTIIHVKGKHLCPSPLFPGPSKPFWVLVVVGGVLACYSLLVTVAFIIFWV

SEQ ID NO: 27 - protein, 6 aa, unidentified
SRNGMS

SEQ ID NO: 28 - protein, 17 aa, unidentified
TVSSGGSYIYYADSVKG

SEQ ID NO: 29 - protein, 13 aa, unidentified
QGTTALATRFFDV

SEQ ID NO: 30 - protein, 11 aa, unidentified
KASQDVGTAVA

SEQ ID NO: 31 - protein, 9 aa, unidentified
QHHYSAPWT

SEQ ID NO: 32 - protein, 122 aa, unidentified
EVQLVESGGGLVQPGGSLRLSCAASGFTFSRNGMSWVRQAPGKGLEWVATVSSGGSYIYYADSVKGRFTISRDNAKNSLYLQMNSLRAEDTAVYYCARQGTTALATRFFDVWGQGTLVTVSS

SEQ ID NO: 33 - protein, 107 aa, unidentified
DIQMTQSPSSLSASVGDRVTITCKASQDVGTAVAWYQQKPGKAPKLLIYSASYRSTGVPSRFSGSGSGTDFTLTISSLQPEDFATYYCQHHYSAPWTFGGGTKVEIK

SEQ ID NO: 34 - protein, 5 aa, unidentified
DYYIH

SEQ ID NO: 35 - protein, 17 aa, unidentified
RIDPENDETKYGPIFQG

SEQ ID NO: 36 - protein, 5 aa, unidentified
RGGVY

SEQ ID NO: 37 - protein, 16 aa, unidentified
KSSQSLLDSDGKTYLN

SEQ ID NO: 38 - protein, 7 aa, unidentified
LVSKLDS

SEQ ID NO: 39 - protein, 9 aa, unidentified
WQGTHFPGT

SEQ ID NO: 40 - protein, 114 aa, unidentified
EIQLQQSGAELVKPGASVKLSCTGSGFNIEDYYIHWVKQRTEQGLEWIGRIDPENDETKYGPIFQGRATITADTSSNTVYLQLSSLTSEDTAVYYCAFRGGVYWGPGTTLTVSS

SEQ ID NO: 41 - protein, 112 aa, unidentified
DVVMTQSPLTLSVAIGQSASISCKSSQSLLDSDGKTYLNWLLQRPGQSPKRLISLVSKLDSGVPDRFTGSGSGTDFTLRISRVEAEDLGIYYCWQGTHFPGTFGGGTKLEIK

SEQ ID NO: 42 - protein, 7 aa, unidentified
GFNIEDY

SEQ ID NO: 43 - protein, 6 aa, unidentified
DPENDE

SEQ ID NO: 44 - protein, 5 aa, unidentified
RGGVY

SEQ ID NO: 45 - protein, 12 aa, unidentified
SQSLLDSDGKTY

SEQ ID NO: 46 - protein, 6 aa, unidentified
GTHFPG

SEQ ID NO: 47 - protein, 114 aa, unidentified; identical to SEQ ID NO: 40

SEQ ID NO: 48 - protein, 112 aa, unidentified; identical to SEQ ID NO: 41

SEQ ID NO: 49 - protein, 5 aa, unidentified
DYYIH

SEQ ID NO: 50 - protein, 17 aa, unidentified
RIDPENDETKYGPIFQG

SEQ ID NO: 51 - protein, 6 aa, unidentified
FRGGVY

SEQ ID NO: 52 - protein, 16 aa, unidentified
KSSQSLLDSDGKTYLN

SEQ ID NO: 53 - protein, 7 aa, unidentified
LVSKLDS

SEQ ID NO: 54 - protein, 9 aa, unidentified
WQGTHFPGT

SEQ ID NO: 55 - protein, 114 aa, unidentified
EIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSS

SEQ ID NO: 56 - protein, 112 aa, unidentified
DVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 57 - protein, 262 aa, unidentified
DIQMTQSPSSLSASVGDRVTITCRASQRASESVDNYGISFMNAWYQQKPGKAPKAASRQGSGSGVPSRFSGSGSGTDFTLTISSLQPEDFATYYCQQSKEVPWTGTKVEIKRTVAAPSGGGGSGGGGSGGGGSEVQLVESGGGLVQPGGSLRLSCAASGFTFSNYLMNWVRQAPGKGLEWVRIDPYDGDIDYNQNFKDGGSTYYADSVKGRFTISADTSKNTAYLQMNSLRAEDTAVYYCAGYGTAYGVDYYWGQGTLVTVS

SEQ ID NO: 58 - protein, 262 aa, unidentified
EVQLVESGGGLVQPGGSLRLSCAASGFTFSNYLMNWVRQAPGKGLEWVRIDPYDGDIDYNQNFKDGGSTYYADSVKGRFTISADTSKNTAYLQMNSLRAEDTAVYYCAGYGTAYGVDYYWGQGTLVTVSGGGGSGGGGSGGGGSDIQMTQSPSSLSASVGDRVTITCRASQRASESVDNYGISFMNAWYQQKPGKAPKAASRQGSGSGVPSRFSGSGSGTDFTLTISSLQPEDFATYYCQQSKEVPWTGTKVEIKRTVAAPS

SEQ ID NO: 59 - protein, 246 aa, unidentified
EIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 60 - protein, 523 aa, unidentified
EIQLVQSGAEVKKPGATVKISCKGSGFNIEDYYIHWVQQAPGKGLEWMGRIDPENDETKYGPIFQGRVTITADTSTNTVYMELSSLRSEDTAVYYCAFRGGVYWGQGTTVTVSSGGGGSGGGGSGGGGSGGGGSDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGGGGSGGGGSGGGGSDIQMTQSPSSLSASVGDRVTITCRASQRASESVDNYGISFMNAWYQQKPGKAPKAASRQGSGSGVPSRFSGSGSGTDFTLTISSLQPEDFATYYCQQSKEVPWTGTKVEIKRTVAAPSGGGGSGGGGSGGGGSEVQLVESGGGLVQPGGSLRLSCAASGFTFSNYLMNWVRQAPGKGLEWVRIDPYDGDIDYNQNFKDGGSTYYADSVKGRFTISADTSKNTAYLQMNSLRAEDTAVYYCAGYGTAYGVDYYWGQGTLVTVS

SEQ ID NO: 61 - protein, 113 aa, unidentified
APGPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPF

SEQ ID NO: 62 - protein, 10 aa, unidentified
GFTFRKFGMS

SEQ ID NO: 63 - protein, 20 aa, unidentified
SISTGGYNTYYSDNVKGRFT

SEQ ID NO: 64 - protein, 11 aa, unidentified
GYSSTSYAMDY

SEQ ID NO: 65 - protein, 11 aa, unidentified
MTSTDIDDDMN

SEQ ID NO: 66 - protein, 7 aa, unidentified
GTDFVFT

SEQ ID NO: 67 - protein, 9 aa, unidentified
LQSFNVPLT

SEQ ID NO: 68 - protein, 140 aa, unidentified
MDWIWRILFLVGAATGAHSQVQLQQSGGGLVKPGASLKLSCVTSGFTFRKFGMSWVRQTSDKRLEWVASISTGGYNTYYSDNVKGRFTISRENAKNTLYLQMSSLKSEDTALYYCTRGYSSTSYAMDYWGQGTTVTVSSS

SEQ ID NO: 69 - protein, 118 aa, unidentified
DIELTQSPASLSVATGEKVTIRCMTSTDIDDDMNWYQQKPGEPPKFLISEGNTLRPGVPSRFSSSGTGTDFVFTIENTLSEDVGDYYCLQSFNVPLTFGDGTKLEKALEQKLISEEDL

SEQ ID NO: 70 - protein, 10 aa, unidentified
GFTFSSYAMS

SEQ ID NO: 71 - protein, 20 aa, unidentified
AISGSGGSTNYADSVKGRFT

SEQ ID NO: 72 - protein, 11 aa, unidentified
RASQGIRNNLA

SEQ ID NO: 73 - protein, 7 aa, unidentified
AASNLQS

SEQ ID NO: 74 - protein, 9 aa, unidentified
LQHHSYPLT

SEQ ID NO: 75 - protein, 119 aa, unidentified
EVQVLESGGGLVQPGGSLRLSCAASGFTFSSYAMSWVRQAPGKGLEWVSAISGSGGSTNYADSVKGRFTISRDNSKNTLYLQMNSLRAEDTAVYYCAGSSGWSEYWGQGTLVTVSSAAA

SEQ ID NO: 76 - protein, 146 aa, unidentified
MVLLVTSLLLCELPHPAFLLIPDIQMTQSPSSLSASVGDRVTITCRASQGIRNNLAWYQQKPGKAPKRLIYAASNLQSGVPSRFTGSGSGTEFTLIVSSLQPEDFATYYCLQHHSYPLTSGGGTKVEIKRTGSTSGSGKPGSGEGS

SEQ ID NO: 77 - protein, 10 aa, unidentified
GFTISTNAMS

SEQ ID NO: 78 - protein, 19 aa, unidentified
VITGRDITYYASWAKGRFT

SEQ ID NO: 79 - protein, 12 aa, unidentified
DGGSSAITSNNI

SEQ ID NO: 80 - protein, 11 aa, unidentified
QASESISSWLA

SEQ ID NO: 81 - protein, 7 aa, unidentified
EASKLAS

SEQ ID NO: 82 - protein, 14 aa, unidentified
QGYFYFISRTYVNS

SEQ ID NO: 83 - protein, 120 aa, unidentified
EVQLVESGGGLVQPGGSLRLSCAASGFTISTNAMSWVRQAPGKGLEWIGVITGRDITYYASWAKGRFTISRDNSKNTLYLQMNSLRAEDTAVYYCARDGGSSAITSNNIWGQGTLVTVSS

SEQ ID NO: 84 - protein, 112 aa, unidentified
DVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGGGTKVEIK

SEQ ID NO: 85 - protein, 11 aa, unidentified
GYSITSDFAWN

SEQ ID NO: 86 - protein, 17 aa, unidentified
YISYSGNTRYNPSLKSR

SEQ ID NO: 87 - protein, 7 aa, unidentified
AGRGFPY

SEQ ID NO: 88 - protein, 11 aa, unidentified
HSSQDINSNIG

SEQ ID NO: 89 - protein, 17 aa, unidentified
YISYSGNTRYNPSLKSR

SEQ ID NO: 90 - protein, 7 aa, unidentified
AGRGFPY

SEQ ID NO: 91 - protein, 116 aa, unidentified
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSA

SEQ ID NO: 92 - protein, 107 aa, unidentified
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIK

SEQ ID NO: 93 - protein, 5 aa, unidentified
DYYIH

SEQ ID NO: 94 - protein, 13 aa, unidentified
RIDPENDETKYGP

SEQ ID NO: 95 - protein, 5 aa, unidentified
RGGVY

SEQ ID NO: 96 - protein, 16 aa, unidentified
KSSQSLLDSDGKTYLN

SEQ ID NO: 97 - protein, 7 aa, unidentified
LVSKLDS

SEQ ID NO: 98 - protein, 9 aa, unidentified
WQGTHFPGT

SEQ ID NO: 99 - protein, 114 aa, unidentified
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSS

SEQ ID NO: 100 - protein, 112 aa, unidentified; identical to SEQ ID NO: 56

SEQ ID NO: 101 - protein, 114 aa, unidentified; identical to SEQ ID NO: 99

SEQ ID NO: 102 - protein, 112 aa, unidentified
DVVMTQSPLSLPVTLGQPASISCKSSQSLLDSDGKTYLNWLQQRPGQSPRRLISLVSKLDSGVPDRFSGSGSGTDFTLKISRVEAEDVGVYYCWQGTHFPGTFGGGTKVEIK

SEQ ID NO: 103 - protein, 114 aa, unidentified; identical to SEQ ID NO: 55

SEQ ID NO: 104 - protein, 112 aa, unidentified; identical to SEQ ID NO: 102

SEQ ID NO: 105 - protein, 114 aa, unidentified; identical to SEQ ID NO: 40

SEQ ID NO: 106 - protein, 114 aa, unidentified
HMDVVMTQSPLTLSVAIGQSASISCKSSQSLLDSDGKTYLNWLLQRPGQSPKRLISLVSKLDSGVPDRFTGSGSGTDFTLRISRVEAEDLGIYYCWQGTHFPGTFGGGTKLEIK

(19) United States
(12) Patent Application Publication
(10) Pub. No.: US 2026/0294969 A1
(43) Pub. Date: Oct. 1, 2026

(54) CAR-T CELLS CO-EXPRESSING CD40L

(71) Applicant: H. LEE MOFFITT CANCER CENTER AND RESEARCH INSTITUTE INC., Tampa, FL (US)

(72) Inventors: Patrick HWU, Tampa, FL (US); Sarah LULU, Tampa, FL (US); Saheed Oluwasina OSENI, Tampa, FL (US)

(21) Appl. No.: 19/475,427

(22) PCT Filed: Apr. 19, 2024
(86) PCT No.: PCT/US2024/025454

§ 371 Date: Oct. 15, 2025

Related U.S. Application Data
(60) Provisional application No. 63/497,090, filed on Apr. 19, 2023.

(51) Int. Cl. A61K 35/17; A61K 38/00; A61K 40/11; A61K 40/31; A61K 40/42; A61K 40/50; C07K 14/705; C07K 16/28; C12N 5/0783; C12N 15/63

(52) U.S. Cl. CPC A61K 35/17; A61K 38/00; A61K 40/11; A61K 40/31; A61K 40/4204; A61K 40/4234; A61K 40/50; C07K 14/7051; C07K 14/70578; C07K 16/2818; C07K 16/2827; C07K 16/2863; C12N 5/0636; C12N 15/63; A61K 2239/47; C07K 2317/31; C07K 2317/53; C07K 2317/565; C07K 2317/622; C07K 2319/02; C07K 2319/03

(57) ABSTRACT

Disclosed are compositions and methods for targeted treatment of glioblastomas. In particular, bi-specific chimeric antigen receptor (CAR) T cells are disclosed that can be used with adoptive cell transfer to target and kill glioblastomas. Therefore, also disclosed are methods of providing an anti-tumor immunity in a subject with glioblastomas that involves adoptive transfer of the disclosed bi-specific CAR T cells.

### CROSS-REFERENCE TO RELATED APPLICATIONS

This application claims benefit of U.S. Provisional Application No. 63/497,090, filed Apr. 19, 2023, which is hereby incorporated herein by reference in its entirety.

### SEQUENCE LISTING

This application contains a sequence listing filed in ST.26 format entitled “320803_2960_Sequence_Listing” created on Apr. 18, 2024, having 98,401 bytes. The content of the sequence listing is incorporated herein in its entirety.

### BACKGROUND OF THE INVENTION

Despite increasing cancer survivorship, the prognosis for patients with Glioblastoma remains poor, with <5% surviving greater than 5 years (Cantrell, J. N., et al., Mayo Clin Proc, 2019. 94 (7): 1278-1286). Major hurdles of CAR-T cell therapy include addressing heterogeneity induced antigen-escape while limiting off-tumor toxicity (Li, L., et al., Front Immunol, 2020. 11:594271).

### SUMMARY OF THE INVENTION

CAR-Ts targeting tumor-specific antigens such as EGFRviii and tumor-associated antigens such as IL13Rα2 have shown promise in human case reports with limited toxicity to normal tissue (Durgin, J. S., et al., Frontiers in Oncology, 2021. 11; Brown, C. E., et al., Mol Ther, 2018. 26 (1): 31-44). Both CAR-T cell targets are currently undergoing further investigation through separate clinical trials (Maggs, L., et al., Frontiers in Neuroscience, 2021. 15). Combining these two targets in tandem, such that CAR-T cells may activate in the presence of one antigen or the other, reduces the risk of antigen-escape. CD40L may further address heterogeneity by activating antigen presenting cells exposed to unknown tumor antigens facilitating adaptive immunity (Elgueta, R., et al., Immunol Rev, 2009. 229 (1): 152-72). Systemic delivery of CD40 agonism in Glioblastoma patients induces meningeal tertiary lymphoid structures formation which correlates with increased tumor T cell infiltration (van Hooren, L., et al., Nature Communications, 2021. 12 (1): 4127). Thus CAR-T cells co-expressing EGFRviii.tandem.IL13Rα2 CAR T receptors and CD40L target a broader population of tumor cells reducing the risk of recurrence compared to single-targeted CAR-T therapy alone.

Therefore, disclosed herein are chimeric antigen receptor (CAR) T cells that can be used with adoptive cell transfer to target and kill glioblastomas. In some embodiments, the CAR T cell is an immune effector cell engineered to express a CD40 agonist (e.g. CD40L polypeptide or anti-CD40 scFv) and/or a Toll-like Receptor (TLR) agonist, and a chimeric antigen receptor polypeptide that selectively binds IL13rα2 and/or EGFRviii. The disclosed bi-specific CAR polypeptides can therefore contain in an ectodomain an antigen-binding domain containing an anti-IL13rα2 binding agent or an anti-EGFRviii binding agent that can bind IL13rα2 or EGFRviii-expressing glioblastoma cells. Also disclosed are bi-specific CAR polypeptides that contain in an ectodomain an antigen-binding domain containing an anti-IL13rα2 binding agent and an anti-EGFRviii binding agent that can bind IL13rα2 and EGFRviii-expressing glioblastoma cells. Also disclosed is an immune effector cell genetically modified to express the disclosed CAR polypeptide.

As with other CARs, the disclosed polypeptides can also contain a transmembrane domain and an endodomain capable of activating an immune effector cell. For example, the endodomain can contain a signaling domain and one or more co-stimulatory signaling regions.

In some embodiments, the intracellular signaling domain is a CD3 zeta (CD3ζ) signaling domain. In some embodiments, the costimulatory signaling region comprises the cytoplasmic domain of CD28, 4-1BB, or a combination thereof. In some cases, the costimulatory signaling region contains 1, 2, 3, or 4 cytoplasmic domains of one or more intracellular signaling and/or costimulatory molecules. In some embodiments, the co-stimulatory signaling region contains one or more mutations in the cytoplasmic domains of CD28 and/or 4-1BB that enhance signaling.

Also disclosed are isolated nucleic acid sequences encoding the disclosed CAR polypeptides, vectors comprising these isolated nucleic acids, cells containing these vectors, and cells comprising one or more of the herein described CAR polypeptides.

In some embodiments, the disclosed CAR T cell exhibits an anti-tumor immunity when the antigen binding domain of a CAR polypeptides binds to CD19 and CD20.

Also disclosed is a method of providing an anti-tumor immunity in a subject with a myeloid or B cell malignancies that involves administering to the subject an effective amount of a CAR T cell disclosed herein. In some cases, the myeloid or B cell malignancies comprises Acute Myeloid Leukemia (AML), blastic plasmocytoid dendritic cell neoplasm, hairy cell leukemia, or Acute Lymphoblastic Leukemia.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIG. 1 shows IL13Rα2 expression in cancers.

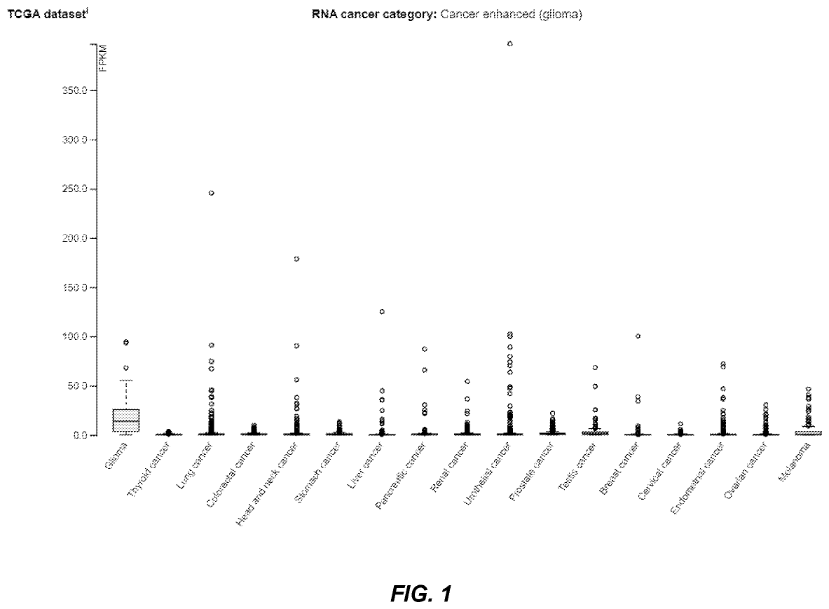

FIG. 2 shows a CD28 second generation CAR-T design where anti-CD19 scFv is replaced with an IL-13 ligand.

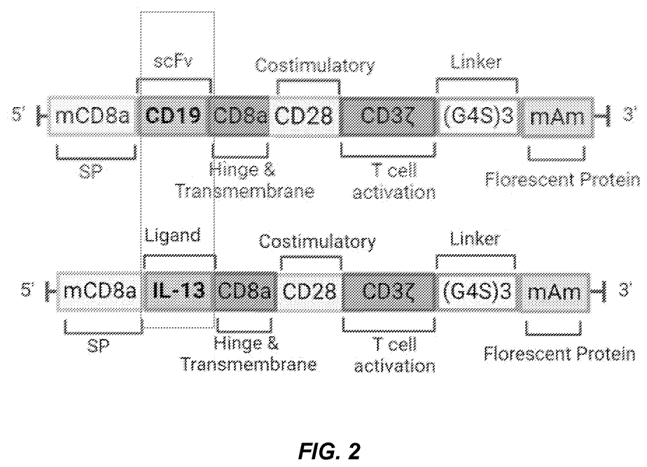

FIG. 3 shows IFNγ levels after co-culture 13.28zReg, 13.BBzReg, 19.28zStbl, 13.28zStbl, and mock CAR-T cells with CD2A.miL-13Rα2.mGFP, CT2A.mGFP (neg-ctrl), and 3T3/CD19+ (pos-ctrl) target cell lines.

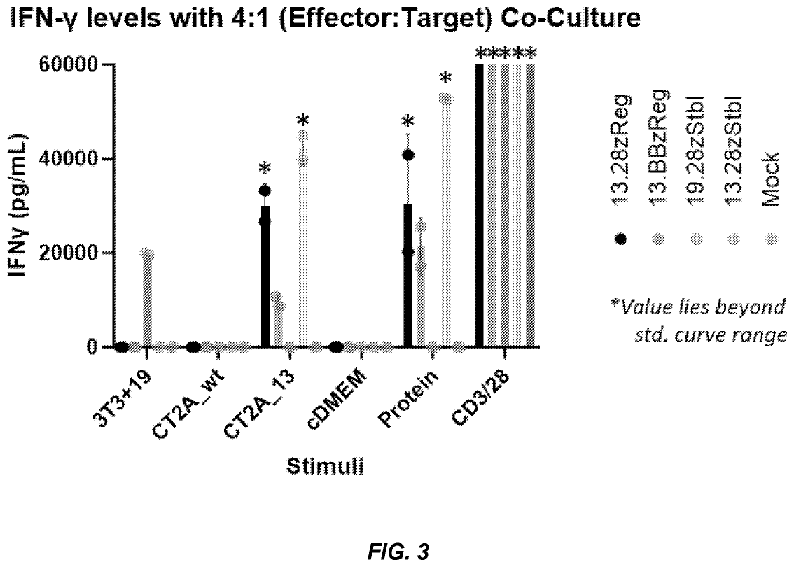

FIG. 4 shows a IL13 ligand CAR construct where CD40 ligand (CD40L) is expressed as an independent peptide (m13.BBz+40L).

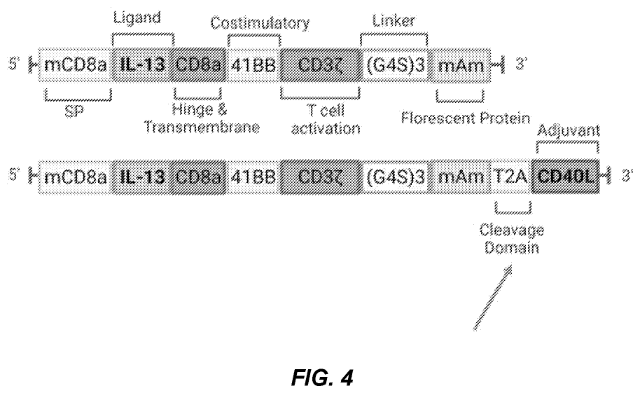

FIGS. 5A and 5B show CD40L increases M1-like marker intensity.

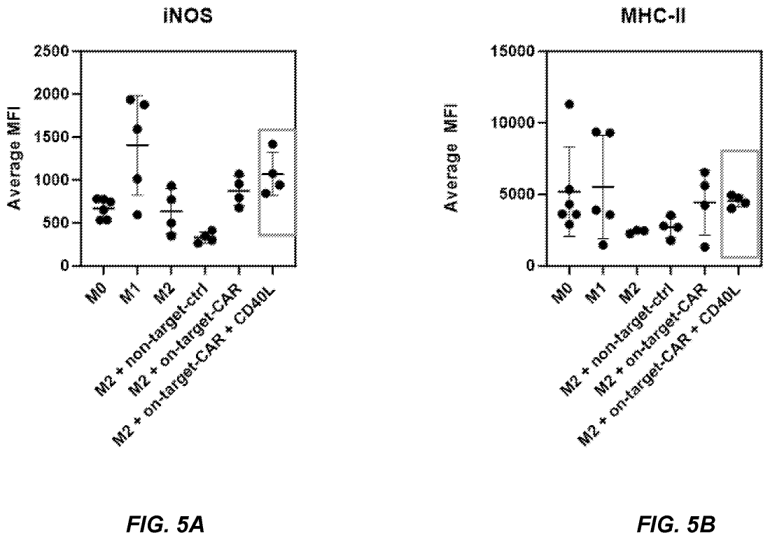

FIG. 6 shows CAR-T driven bone marrow derived macrophages. There was an upregulation of iNOS in M2 polarized macrophages exposed to CAR-T+CD40L.

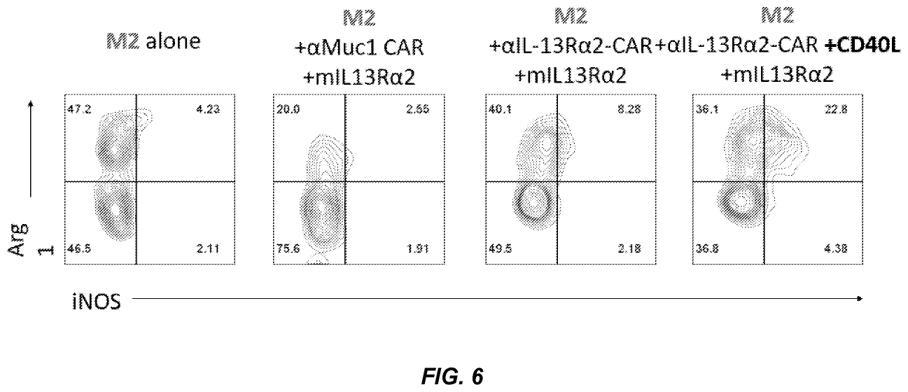

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class. In addition to intact immunoglobulin molecules, also included in the term “antibodies” are fragments or polymers of those immunoglobulin molecules, and human or humanized versions of immunoglobulin molecules that selectively bind the target antigen.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)2, scFv, Fv, dsFv diabody, Fc, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “antigen binding site” refers to a region of an antibody that specifically binds an epitope on an antigen.

The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “chimeric molecule” refers to a single molecule created by joining two or more molecules that exist separately in their native state. The single, chimeric molecule has the desired functionality of all of its constituent molecules. One type of chimeric molecules is a fusion protein.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (for example IgA, IgD, IgE, IgG, IgM and IgY).

The term “epitope” refers to the region of an antigen to which an antibody binds preferentially and specifically. A monoclonal antibody binds preferentially to a single specific epitope of a molecule that can be molecularly defined. In the present invention, multiple epitopes can be recognized by a multispecific antibody.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “Fab fragment” refers to a fragment of an antibody comprising an antigen-binding site generated by cleavage of the antibody with the enzyme papain, which cuts at the hinge region N-terminally to the inter-H-chain disulfide bond and generates two Fab fragments from one antibody molecule.

The term “F(ab′)2 fragment” refers to a fragment of an antibody containing two antigen-binding sites, generated by cleavage of the antibody molecule with the enzyme pepsin which cuts at the hinge region C-terminally to the inter-H-chain disulfide bond.

The term “Fc fragment” refers to the fragment of an antibody comprising the constant domain of its heavy chain.

The term “Fv fragment” refers to the fragment of an antibody comprising the variable domains of its heavy chain and light chain.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “multivalent antibody” refers to an antibody or engineered antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “peptide,” “protein,” and “polypeptide” are used interchangeably to refer to a natural or synthetic molecule comprising two or more amino acids linked by the carboxyl group of one amino acid to the alpha amino group of another.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “protein domain” refers to a portion of a protein, portions of a protein, or an entire protein showing structural integrity; this determination may be based on amino acid composition of a portion of a protein, portions of a protein, or the entire protein.

The term “single chain variable fragment or scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

A “spacer” as used herein refers to a peptide that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “transformation” and “transfection” mean the introduction of a nucleic acid, e.g., an expression vector, into a recipient cell including introduction of a nucleic acid to the chromosomal DNA of said cell.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “variant” refers to an amino acid or peptide sequence having conservative amino acid substitutions, non-conservative amino acid substitutions (i.e. a degenerate variant), substitutions within the wobble position of each codon (i.e. DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

The term “vector” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element).

### CD40 or TLR Agonists

In some embodiments, the disclosed bi-specific CAR T cell is an immune effector cell engineered to co-express the CAR polypeptide with a CD40 agonist.

In some embodiments, the CD40 agonist is a CD40L polypeptide. For example, in some embodiments, the CD40L polypeptide comprises the amino acid sequence

(SEQ ID NO: 22)
MIETYNQTSPRSAATGLPISMKIFMYLLTVFLITQMIGSALFAVYLHRRL
DKIEDERNLHEDFVFMKTIQRCNTGERSLSLLNCEEIKSQFEGFVKDIML
NKEETKKENSFEMQKGDQNPQIAAHVISEASSKTTSVLQWAEKGYYTMSN
NLVTLENGKQLTVKRQGLYYIYAQVTFCSNREASSQAPFIASLCLKSPGR
FERILLRAANTHSSAKPCGQQSIHLGGVFELQPGASVFVNVTDPSQVSHG
TGFTSFGLLKL.

In some embodiments, the CD40 agonist is an antibody that binds CD40, such an scFv that binds CD40.

In some embodiments, the disclosed bi-specific CAR T cell is an immune effector cell engineered to co-express the CAR polypeptide with a Toll-like receptor (TLR) agonist or inflammasomes. For example, in some embodiments, the TLR agonist is Antiphospholipid antibodies (TLR 2/4/7/8 agonist). In some embodiments, the TLR agonist is Bacillus Calmette-Guerin (BCG) (TLR 2/4/9 agonist). In some embodiments, the TLR agonist is β-defensin-3 (TLR 1/2/4 agonist). In some embodiments, the TLR agonist is Gardiquimod (TLR 7/8 agonist). In some embodiments, the TLR agonist or inflammasome is HMGB1 (TLR 2/4 agonist and inflammasome). In some embodiments, the TLR agonist is HSP60 (TLR 2/4 agonist). In some embodiments, the TLR agonist is HSP70 (TLR 2/4 agonist). In some embodiments, the TLR agonist is Hyaluronic acid fragments (TLR 2/4 agonist). In some embodiments, the TLR agonist is synthetic Imidazoquinoline derivatives such as Imidazole (TLR 7/8 agonist). In some embodiments, the TLR agonists is a Nucleoside analog (TLR 7/8 agonist). In some embodiments, the TLR agonist or inflammasome is Profilin from Toxoplasma gondii (TLR 11/12 agonist and inflammasome). In some embodiments, the TLR agonist is Resiquimod (R-848) (TLR 7/8 agonist). In some embodiments, the TLR agonist is viral and bacterial ssRNA and phagocytized bacterial RNA (TLR 7/8 agonist). In some embodiments, the TLR agonist is Triacylated lipoprotein from bacteria (TLR 1/10 agonist).

In some embodiments, the TLR agonist is Atypical bacterial lipopolysaccharides (LPS) (TLR 1 agonist).

In some embodiments, the TLR agonist is Biglycan (TLR 2 agonist). In some embodiments, the TLR agonist is CU-T12-9 (TLR 2 agonist). In some embodiments, the TLR agonist is Eosinophil-de-rived neurotoxins (TLR 2 agonist). In some embodiments, the TLR agonist is Glycoprotein from Treponema maltophilium (TLR 2 agonist). In some embodiments, the TLR agonist is lipoteichoic acid (LTA) from Gram-positive bacterial (TLR 2 agonist). In some embodiments, the TLR agonist is GP96 (TLR 2 agonist). In some embodiments, the TLR agonist is the GPI anchor from Trypanosoma cruzi and Plasmodium falciparum (TLR 2 agonist). In some embodiments, the TLR agonist is HSP90 (TLR 2 agonist). In some embodiments, the TLR agonist is Peptidoglycans (PDG) from Gram-positive bacteria (TLR 2 agonist). In some embodiments, the TLR agonist is Phenol-soluble modulin from Staphylococcus aureus (TLR 2 agonist). In some embodiments, the TLR agonist is Phospho-lipomannan from Candida albicans (TLR 2 agonist). In some embodiments, the TLR agonist is Serum amyloid (TLR 2 agonist). In some embodiments, the TLR agonist is Surfactant protein A/D (TLR 2 agonist). In some embodiments, the TLR agonist is Synthetic bacterial lipoprotein (Pam3CSK4) (TLR 2 agonist). In some embodiments, the TLR agonist is versican (TLR 2 agonist). In some embodiments, the TLR agonist is bacterial Zymosan (TLR 2 agonist).

In some embodiments, the TLR agonists or inflammasomes are Viral double-stranded RNA (dsRNA) and Synthetic dsRNA analogs such as Polyriboinosinic-polyribocytidylic acid Poly(I:C) and Poly(A:U) (TLR 3 agonist).

In some embodiments, the TLR agonist is Fibronectin EDA (TLR 4 agonist). In some embodiments, the TLR agonist is Fibrinogen (TLR 4 agonist). In some embodiments, the TLR agonist is Glucuronoxylomannan from Cryptococcus neoformans (TLR 4 agonist). In some embodiments, the TLR agonist is GP96 (TLR 4 agonist). In some embodiments, the TLR agonist is Heparan sulfate fragments (TLR 4 agonist). In some embodiments, the TLR agonist is HSP22 (TLR 4 agonist). In some embodiments, the TLR agonist is Lactoferrin (TLR 4 agonist). In some embodiments, the TLR agonist is oxidized LDL (TLR 4 agonist). In some embodiments, the TLR agonist or inflammasome is Lipopolysaccharides (LPS) from Gram-negative enterobacteria (TLR 4 agonist and inflammasome). In some embodiments, the TLR agonist is Mannan from Saccharomyces cerevisiae and Candida albicans (TLR 4 agonist). In some embodiments, the TLR agonist is Monophosphoryl lipid A (MPL) (TLR 4 agonist). In some embodiments, the TLR agonist is Neutrophil elastase (TLR 4 agonist). In some embodiments, the TLR agonist is Nickel (TLR 4 agonist). In some embodiments, the TLR agonist is an Opioid receptor μ, δ, and κ agonist (TLR 4 agonist). In some embodiments, the TLR agonist is S100A8 MRP8 (TLR 4 agonist). In some embodiments, the TLR agonist is S100A9 MRP14 (TLR 4 agonist). In some embodiments, the TLR agonist is Saturated fatty acids (TLR 4 agonist). In some embodiments, the TLR agonist is Serum amyloid A (TLR 4 agonist). In some embodiments, the TLR agonist is Surfactant protein A/D (TLR 4 agonist). In some embodiments, the TLR agonist is Tenascin-C (TLR 4 agonist).

In some embodiments, the TLR agonist or inflammasome is flagellin derived from flagellated bacteria such as Salmonella typhimuriam, Pseudomonas aeruginosa, Legionella pneumophilia, and Shigella flexneri (TLR 5 agonist and inflammasome).

In some embodiments, the TLR agonist is Diacetylated lipopeptides (MALP-2) from Mycoplasma sp. (TLR 6 agonist). In some embodiments, the TLR agonist is Triacylated lipopeptides of bacteria (TLR 6 agonist).

In some embodiments, the TLR agonist is Bropirimine (TLR 7 agonist). In some embodiments, the TLR agonist is Imiquimod (TLR 7 agonist).

In some embodiments, the TLR agonist is unmethylated CpG oligodeoxynucleotide DNA of bacteria and viruses, CpG ODN (TLR9 agonist). In some embodiments, the TLR agonist is Hemozoin from Plasmodium falciparum (TLR 9 agonist). In some embodiments, the TLR agonist is IgG-chromatin complexes (TLR 9 agonist).

In some embodiments, the TLR agonist is bacterial ribosomal RNA or rRNA (TLR 13 agonist).

In some embodiments, the TLR agonist is a bacterial cell wall inhibitor polypeptide (TLR 14 agonist).

In some embodiments, the inflammasome is Alum. In some embodiments, the inflammasome is Amyloid B. In some embodiments, the inflammasome is Asbestos. In some embodiments, the inflammasome is lethal bacterial toxin from Bacillus anthracis. In some embodiments, the inflammasome is calreticulin. In some embodiments, the inflammasome is cathepsins. In some embodiments, the inflammasome is Cholesterol crystals. In some embodiments, the inflammasome is Extracellular ATPs. In some embodiments, the inflammasome is Hyaluronan. In some embodiments, the inflammasome is Mitochondrial DNA or mtDNA. In some embodiments, the inflammasome is Mitochondrial ROS or mtROS. In some embodiments, the inflammasome is Monosodium urate. In some embodiments, the inflammasome is bacterial muramyl dipeptides. In some embodiments, the inflammasome is bacterial pore-forming toxins. In some embodiments, the inflammasome is Rho GTPase modifying toxins. In some embodiments, the inflammasome is Silica. In some embodiments, the inflammasome is Uric acid.

In some embodiments agonizing Toll-like receptors initiates an innate immune response against invading pathogens. This can assist in attacking tumor cells not recognized by the CAR-T cell, particularly for heterogenous tumors like GBM, protecting against antigen escape.

### Chimeric Antigen Receptors (CAR)

Disclosed herein is a chimeric antigen receptor (CAR) polypeptide that can be that can be expressed in immune effector cells to enhance antitumor activity against myeloid or B cell malignancies.

The disclosed CAR is generally made up of three domains: an ectodomain, a transmembrane domain, and an endodomain. The disclosed bi-specific ectodomain comprises a IL13rα2-binding region and a EGFRviii-binding region and is responsible for antigen recognition. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

The antigen binding agent is in some embodiments an IL-13 ligand. For example, in some embodiments, the IL-13 ligand has the amino acid sequence: APGPVPRSVSLPLTLKELIEELSNITQDQTPLCNGSMVWSVDLAAGGFCVALDSLTNISNCNAIY RTQRILHGLCNRKAPTTVSSLPDTKIEVAHFITKLLSYTKQLFRHGPF (SEQ ID NO:61), or a human homologue or variant thereof having at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO:61.

The antigen binding agent is in some embodiments an antibody fragment that specifically binds IL13rα2 or EGFRviii. For example, the antigen binding domain can be a Fab or a single-chain variable fragment (scFv) of an antibody that specifically binds IL13rα2 or EGFRviii. The antigen binding agent is in some embodiments an aptamer that specifically binds IL13rα2 or EGFRviii. For example, the antigen binding agent can be a peptide aptamer selected from a random sequence pool based on its ability to bind IL13rα2 or EGFRviii. The antigen binding agent can also be a natural ligand of IL13rα2 or EGFRviii, or a variant and/or fragment thereof capable of binding IL13rα2 or EGFRviii.

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

In some embodiments, the endodomain contains an SD or a CSR, but not both. In these embodiments, an immune effector cell containing the disclosed CAR is only activated if another CAR (or a T-cell receptor) containing the missing domain also binds its respective antigen.

In some embodiments, the disclosed CAR is defined by the formula:

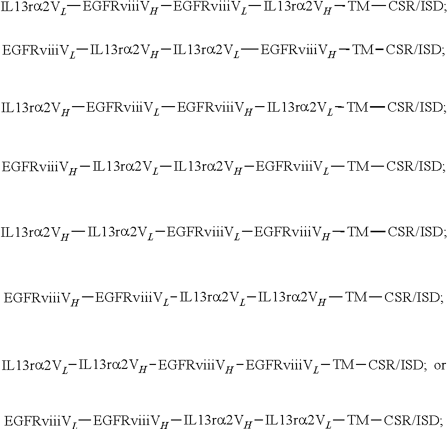

wherein “IL13rα2V_{H}” represents an anti-IL13rα2V_{H} domain,
wherein “IL13rα2V_{L}” represents an anti-IL13rα2V_{L} domain,
wherein “EGFRviiiV_{H}” represents an anti-EGFRviii V_{H} domain,
wherein “EGFRviiiV_{L}” represents an anti-EGFRviii V_{L} domain,
wherein “TM” represents a transmembrane domain,
wherein “CSR/ISD” represents a co-stimulatory signaling region and/or intracellular signaling domain, and
wherein “-” represents a peptide bond or linker.

Anti-IL13rα2 scFv sequences are described in WO2016123142A1, which is incorporated by reference for the teaching of these antibody sequences. In some embodiments, the anti-IL13rα2 scFv can comprise a variable heavy (VA) domain having CDR1, CDR2 and CDR3 sequences and a variable light (V_{L}) domain having CDR1, CDR2 and CDR3 sequences. In some embodiments, the CDR1 sequence of the V_{H} domain comprises the amino acid sequence NYLMN (SEQ ID NO: 1); the CDR2 sequence of the V_{H} domain comprises the amino acid sequence RIDPYDGDIDYNQNFKD (SEQ ID NO:2); the CDR3 sequence of the V_{H} domain comprises the amino acid sequence GYGTAYGVDY (SEQ ID NO:3); the CDR1 sequence of the V_{L} comprises the amino acid sequence RASESVDNYGISFMN (SEQ ID NO:4); the CDR2 sequence of the V_{L} domain comprises the amino acid sequence AASRQGSG (SEQ ID NO:5); and the CDR3 sequence of the V_{L} domain comprises the amino acid sequence QQSKEVPWT (SEQ ID NO:6).

Therefore, in some embodiments, the anti-IL13rα2V_{H} domain comprises the amino acid sequence EVOLVESGGGLVQPGGSLRLSCAASGFTFSNYLMNWVRQAPGKGLEWVRIDPYDGDIDYNQ NFKDGGSTYYADSVKGRFTISADTSKNTAYLQMNSLRAEDTAVYYCAGYGTAYGVDYYWGQG TLVTVS (SEQ ID NO:7); and the anti-IL13rα2 scFv V_{L} domain comprises the amino acid sequence

(SEQ ID NO: 8)
DIQMTQSPSSLSASVGDRVTITCRASQRASESVDNYGISFMNAWYQQKPG
KAPKAASRQGSGSGVPSRFSGSGSGTDFTLTISSLQPEDFATYYCQQSKE
VPWTGTKVEIKRTVAAPS.

In some embodiments, the CDR1 sequence of the V_{H} domain comprises the amino acid sequence SRNGMS (SEQ ID NO:27); the CDR2 sequence of the V_{H} domain comprises the amino acid sequence TVSSGGSYIYYADSVKG (SEQ ID NO:28); the CDR3 sequence of the V_{H} domain comprises the amino acid sequence QGTTALATRFFDV (SEQ ID NO: 29); the CDR1 sequence of the V_{L} comprises the amino acid sequence KASQDVGTAVA (SEQ ID NO:30); and the CDR3 sequence of the V_{L} domain comprises the amino acid sequence QHHYSAPWT (SEQ ID NO:31).

Therefore, in some embodiments, the anti-IL13rα2V_{H} domain comprises the amino acid sequence:

(SEQ ID NO: 32)
EVQLVESGGGLVQPGGSLRLSCAASGFTFSRNGMSWVRQAPGKGLEWVAT
VSSGGSYIYYADSVKGRFTISRDNAKNSLYLQMNSLRAEDTAVYYCARQG
TTALATRFFDVWGQGTLVTVSS;
and
the anti-IL13rα2 scFv V_{L} domain comprises the
amino acid sequence:
(SEQ ID NO: 33)
DIQMTQSPSSLSASVGDRVTITCKASQDVGTAVAWYQQKPGKAPKLLIYS
ASYRSTGVPSRFSGSGSGTDFTLTISSLQPEDFATYYCQHHYSAPWTFGG
GTKVEIK.

In some embodiments, the anti-EGFRviii scFv can comprise a variable heavy (V_{H}) domain having CDR1, CDR2 and CDR3 sequences and a variable light (V_{L}) domain having CDR1, CDR2 and CDR3 sequences. In some embodiments, the CDR1 sequence of the V_{H} domain comprises the amino acid sequence DSWIH (SEQ ID NO:9); the CDR2 sequence of the V_{H} domain comprises the amino acid sequence AWISPY (SEQ ID NO: 10); the CDR3 sequence of the V_{H} domain comprises the amino acid sequence RRHWPGGFD (SEQ ID NO: 11); the CDR1 sequence of the V_{L} comprises the amino acid sequence DVSTAV (SEQ ID NO: 12); the CDR2 sequence of the V_{L} domain comprises the amino acid sequence LLIYSASFLY (SEQ ID NO: 13); and the CDR3 sequence of the V_{L} domain comprises the amino acid sequence QQYLYHPATFGQ (SEQ ID NO:14).

Therefore, in some embodiments, the anti-EGFRviii V_{H} domain comprises the amino acid sequence EVOLVESGGGLVQPGGSLRLSCAASGFTFSDSWIHWVRQAPGKGLEWVAWISPYGGSTYYA DSVKGRFTISADTSKNTAYLQMNSLRAEDTAVYYCARRHWPGGFDYWGQGTLVTVS (SEQ ID NO: 15); and the anti-EGFRviii V_{L} domain comprises the amino acid sequence

(SEQ ID NO: 16)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPS.

Anti-EGFRviii scFv sequences are described in KR102313997B1, which is incorporated by reference for the teaching of these antibody sequences. In some embodiments, the anti-EGFRviii scFv can comprise a variable heavy (V_{H}) domain having CDR1, CDR2 and CDR3 sequences and a variable light (V_{L}) domain having CDR1, CDR2 and CDR3 sequences. In some embodiments, the CDR1 sequence of the V_{H} domain comprises the amino acid sequence DYYIH (SEQ ID NO:34); the CDR2 sequence of the V_{H} domain comprises the amino acid sequence RIDPENDETKYGPIFQG (SEQ ID NO:35); the CDR3 sequence of the V_{H} domain comprises the amino acid sequence RGGVY (SEQ ID NO:36); the CDR1 sequence of the V_{L} comprises the amino acid sequence KSSQSLLDSDGKTYLN (SEQ ID NO:37); the CDR2 sequence of the V_{L} domain comprises the amino acid sequence LVSKLDS (SEQ ID NO:38); and the CDR3 sequence of the V_{L} domain comprises the amino acid sequence WQGTHFPGT (SEQ ID NO:39).

Therefore, in some embodiments, the anti-EGFRviii V_{H} domain comprises the amino acid sequence

(SEQ ID NO: 40)
EIQLQQSGAELVKPGASVKLSCTGSGFNIEDYYIHWVKQRTEQGLEWIGR
IDPENDETKYGPIFQGRATITADTSSNTVYLQLSSLTSEDTAVYYCAFRG
GVYWGPGTTLTVSS;

and the anti-EGFRviii V_{L} domain comprises the amino acid sequence

(SEQ ID NO: 41)
DVVMTQSPLTLSVAIGQSASISCKSSQSLLDSDGKTYLNWLLQRPGQSP
KRLISLVSKLDSGVPDRFTGSGSGTDFTLRISRVEAEDLGIYYCWQGTH
FPGTFGGGTKLEIK.

In some embodiments, the anti-EGFRviii scFv can comprise a variable heavy (V_{H}) domain having CDR1, CDR2 and CDR3 sequences and a variable light (V_{L}) domain having CDR1, CDR2 and CDR3 sequences. In some embodiments, the CDR1 sequence of the V_{H} domain comprises the amino acid sequence GFNIEDY (SEQ ID NO:42); the CDR2 sequence of the V_{H} domain comprises the amino acid sequence DPENDE (SEQ ID NO:43); the CDR3 sequence of the V_{H} domain comprises the amino acid sequence RGGVY (SEQ ID NO:44); the CDR1 sequence of the V_{L} comprises the amino acid sequence SQSLLDSDGKTY (SEQ ID NO: 45); the CDR2 sequence of the V_{L} domain comprises the amino acid sequence LVS; and the CDR3 sequence of the V_{L} domain comprises the amino acid sequence GTHFPG (SEQ ID NO: 46).

Therefore, in some embodiments, the anti-EGFRviii V_{H} domain comprises the amino acid sequence

(SEQ ID NO: 47)
EIQLQQSGAELVKPGASVKLSCTGSGFNIEDYYIHWVKQRTEQGLEWIG
RIDPENDETKYGPIFQGRATITADTSSNTVYLQLSSLTSEDTAVYYCAF
RGGVYWGPGTTLTVSS;

and the anti-EGFRviii V_{L} domain comprises the amino acid sequence

(SEQ ID NO: 48)
DVVMTQSPLTLSVAIGQSASISCKSSQSLLDSDGKTYLNWLLQRPGQSP
KRLISLVSKLDSGVPDRFTGSGSGTDFTLRISRVEAEDLGIYYCWQGTH
FPGTFGGGTKLEIK.

Anti-EGFRviii scFv sequences are described in WO2014130657A1, which is incorporated by reference for the teaching of these antibody sequences. In some embodiments, the anti-anti-EGFRviii scFv can comprise a variable heavy (V_{H}) domain having CDR1, CDR2 and CDR3 sequences and a variable light (V_{L}) domain having CDR1, CDR2 and CDR3 sequences. In some embodiments, the CDR1 sequence of the V_{H} domain comprises the amino acid sequence DYYIH (SEQ ID NO:49); the CDR2 sequence of the V_{H} domain comprises the amino acid sequence RIDPENDETKYGPIFQG (SEQ ID NO:50); the CDR3 sequence of the V_{H} domain comprises the amino acid sequence FRGGVY (SEQ ID NO:51); the CDR1 sequence of the V_{L} comprises the amino acid sequence KSSQSLLDSDGKTYLN (SEQ ID NO:52); the CDR2 sequence of the V_{L} domain comprises the amino acid sequence LVSKLDS (SEQ ID NO:53); and the CDR3 sequence of the V_{L} domain comprises the amino acid sequence WQGTHFPGT (SEQ ID NO:54).

Therefore, in some embodiments, the anti-EGFRviii V_{H} domain comprises the amino acid sequence

(SEQ ID NO: 55)
EIQLVQSGAEVKKPGATVKISCKGSGFNIEDYYIHWVQQAPGKGLEWMG
RIDPENDETKYGPIFQGRVTITADTSTNTVYMELSSLRSEDTAVYYCAF
RGGVYWGQGTTVTVSS;

the anti-EGFRviii scFv V_{L} domain comprises the amino acid sequence

(SEQ ID NO: 56)
DVVMTQSPDSLAVSLGERATINCKSSQSLLDSDGKTYLNWLQQKPGQPP
KRLISLVSKLDSGVPDRFSGSGSGTDFTLTISSLQAEDVAVYYCWQGTH
FPGTFGGGTKVEIK.

Anti-EGFRviii scFv sequences are also described in US20100105136A1, which is incorporated by reference for the teaching of these antibody sequences. In some embodiments, the anti-EGFRviii scFv can comprise a variable heavy (VA) domain having CDR1, CDR2 and CDR3 sequences and a variable light (V_{L}) domain having CDR1, CDR2 and CDR3 sequences. In some embodiments, the CDR1 sequence of the V_{H} domain comprises the amino acid sequence GFTFRKFGMS (SEQ ID NO:62); the CDR2 sequence of the V_{H} domain comprises the amino acid sequence SISTGGYNTYYSDNVKGRFT (SEQ ID NO:63); the CDR3 sequence of the V_{H} domain comprises the amino acid sequence GYSSTSYAMDY (SEQ ID NO: 64); the CDR1 sequence of the V_{L} comprises the amino acid sequence MTSTDIDDDMN (SEQ ID NO:65); the CDR2 sequence of the V_{L} domain comprises the amino acid sequence GTDFVFT (SEQ ID NO:66); and the CDR3 sequence of the V_{L} domain comprises the amino acid sequence LQSFNVPLT (SEQ ID NO:67).

Therefore, in some embodiments, the anti-EGFRviii V_{H} domain comprises the amino acid sequence MDWIWRILFLVGAATGAHSQVQLQQSGGGLVKPGASLKLSCVTSGFTFRKFGMSWVRQTSDK RLEWVASISTGGYNTYYSDNVKGRFTISRENAKNTLYLQMSSLKSEDTALYYCTRGYSSTSYA MDYWGQGTTVTVSSS (SEQ ID NO:68); and the anti-EGFRviii V_{L} domain comprises the amino acid sequence

(SEQ ID NO: 69)
DIELTQSPÅSLSVATGEKVTIRCMTSTDIDDDMNWYQQKPGEPPKFLIS
EGNTLRPGVPSRFSSSGTGTDFVFTIENTLSEDVGDYYCLQSFNVPLTF
GDGTKLEKALEQKLISEEDL.

Anti-EGFRviii scFv sequences are also described in U.S. Pat. No. 11,879,017, which is incorporated by reference for the teaching of these antibody sequences. In some embodiments, the anti-EGFRviii scFv can comprise a variable heavy (V_{H}) domain having CDR1, CDR2 and CDR3 sequences and a variable light (V_{L}) domain having CDR1, CDR2 and CDR3 sequences. In some embodiments, the CDR1 sequence of the V_{H} domain comprises the amino acid sequence GFTFSSYAMS (SEQ ID NO:70); the CDR2 sequence of the V_{H} domain comprises the amino acid sequence AISGSGGSTNYADSVKGRFT (SEQ ID NO:71); the CDR3 sequence of the V_{H} domain comprises the amino acid sequence SSG; the CDR1 sequence of the V_{L} comprises the amino acid sequence RASQGIRNNLA (SEQ ID NO:72); the CDR2 sequence of the V_{L} domain comprises the amino acid sequence AASNLQS (SEQ ID NO:73); and the CDR3 sequence of the V_{L} domain comprises the amino acid sequence LQHHSYPLT (SEQ ID NO:74).

Therefore, in some embodiments, the anti-EGFRviii V_{H} domain comprises the amino acid sequence EVQVLESGGGLVQPGGSLRLSCAASGFTFSSYAMSWVRQAPGKGLEWVSAISGSGGSTNYA DSVKGRFTISRDNSKNTLYLQMNSLRAEDTAVYYCAGSSGWSEYWGQGTLVTVSSAAA (SEQ ID NO: 75); and the anti-EGFRviii V_{L} domain comprises the amino acid sequence

(SEQ ID NO: 76)
MVLLVTSLLLCELPHPAFLLIPDIQMTQSPSSLSASVGDRVTITCRASQ
GIRNNLAWYQQKPGKAPKRLIYAASNLQSGVPSRFTGSGSGTEFTLIVS
SLQPEDFATYYCLQHHSYPLTSGGGTKVEIKRTGSTSGSGKPGSGEGS.

In some embodiments, the anti-EGFRviii scFv can be derived from the tetraspecific humanized anti-EGFRviii antibody Umizortamig. In some embodiments, the anti-EGFRviii scFv can comprise a variable heavy (V_{H}) domain having CDR1, CDR2 and CDR3 sequences and a variable light (V_{L}) domain having CDR1, CDR2 and CDR3 sequences. In some embodiments, the CDR1 sequence of the V_{H} domain comprises the amino acid sequence GFTISTNAMS (SEQ ID NO:77); the CDR2 sequence of the V_{H} domain comprises the amino acid sequence VITGRDITYYASWAKGRFT (SEQ ID NO:78); the CDR3 sequence of the V_{H} domain comprises the amino acid sequence DGGSSAITSNNI (SEQ ID NO:79); the CDR1 sequence of the V_{L} comprises the amino acid sequence QASESISSWLA (SEQ ID NO:80); the CDR2 sequence of the V_{L} domain comprises the amino acid sequence EASKLAS (SEQ ID NO: 81); and the CDR3 sequence of the V_{L} domain comprises the amino acid sequence QGYFYFISRTYVNS (SEQ ID NO:82).

Therefore, in some embodiments, the anti-EGFRviii V_{H} domain comprises the amino acid sequence EVQLVESGGGLVQPGGSLRLSCAASGFTISTNAMSWVRQAPGKGLEWIGVITGRDITYYASWA KGRFTISRDNSKNTLYLQMNSLRAEDTAVYYCARDGGSSAITSNNIWGQGTLVTVSS (SEQ ID NO: 83); and the anti-EGFRviii V_{L} domain comprises the amino acid sequence

(SEQ ID NO: 84)
DVVMTQSPSTLSASVGDRVTINCQASESISSWLAWYQQKPGKAPKLLIY
EASKLASGVPSRFSGSGSGTEFTLTISSLQPDDFATYYCQGYFYFISRT
YVNSFGGGTKVEIK.

In some embodiments, the anti-EGFRviii scFv can comprise a variable heavy (V_{H}) domain having CDR1, CDR2 and CDR3 sequences and a variable light (V_{L}) domain having CDR1, CDR2 and CDR3 sequences. In some embodiments, the CDR1 sequence of the V_{H} domain comprises the amino acid sequence GYSITSDFAWN (SEQ ID NO:85); the CDR2 sequence of the V_{H} domain comprises the amino acid sequence YISYSGNTRYNPSLKSR (SEQ ID NO: 86); the CDR3 sequence of the V_{H} domain comprises the amino acid sequence AGRGFPY (SEQ ID NO:87); the CDR1 sequence of the V_{L} comprises the amino acid sequence HSSQDINSNIG (SEQ ID NO:88); the CDR2 sequence of the V_{L} domain comprises the amino acid sequence YISYSGNTRYNPSLKSR (SEQ ID NO:89); and the CDR3 sequence of the V_{L} domain comprises the amino acid sequence AGRGFPY (SEQ ID NO:90).

Therefore, in some embodiments, the anti-EGFRviii V_{H} domain comprises the amino acid sequence:

(SEQ ID NO: 91)
DVQLQESGPSLVKPSQSLSLTCTVTGYSITSDFAWNWIRQFPGNKLEWM
GYISYSGNTRYNPSLKSRISITRQTSKNQFFLQLNSVTIEDTATYYCVT
AGRGFPYWGQGTLVTVSA;

and the anti-EGFRviii V_{L} domain comprises the amino acid sequence

(SEQ ID NO: 92)
DILMTQSPSSMSVSLGDTVSITCHSSQDINSNIGWLQQRPGKSFKGLIY
HGTNLDDEVPSRFSGSGSGADYSLTISSLESEDFADYYCVQYAQFPWTF
GGGTKLEIK.

Anti-EGFRviii scFv sequences are also described in U.S. Pat. No. 11,865,167, which is incorporated by reference for the teaching of these antibody sequences. In some embodiments, the anti-EGFRviii scFv can comprise a variable heavy (V_{H}) domain having CDR1, CDR2 and CDR3 sequences and a variable light (V_{L}) domain having CDR1, CDR2 and CDR3 sequences. In some embodiments, the CDR1 sequence of the V_{H} domain comprises the amino acid sequence DYYIH (SEQ ID NO:93); the CDR2 sequence of the V_{H} domain comprises the amino acid sequence RIDPENDETKYGP (SEQ ID NO:94); the CDR3 sequence of the V_{H} domain comprises the amino acid sequence RGGVY (SEQ ID NO:95); the CDR1 sequence of the V_{L} comprises the amino acid sequence KSSQSLLDSDGKTYLN (SEQ ID NO:96); the CDR2 sequence of the V_{L} domain comprises the amino acid sequence LVSKLDS (SEQ ID NO:97); and the CDR3 sequence of the V_{L} domain comprises the amino acid sequence WQGTHFPGT (SEQ ID NO:98).

Therefore, in some embodiments, the anti-EGFRviii V_{H} domain comprises the amino acid sequence

(SEQ ID NO: 99)
EIQLVQSGAEVKKPGESLRISCKGSGFNIEDYYIHWVRQMPGKGLEWMG
RIDPENDETKYGPIFQGHVTISADTSINTVYLQWSSLKASDTAMYYCAF
RGGVYWGQGTTVTVSS;

and the anti-EGFRviii V_{L} domain comprises the amino acid sequence

(SEQ ID NO: 100)
DVVMTQSPDSLAVSLGERATINCKSSQSLLDSDGKTYLNWLQQKPGQPP
KRLISLVSKLDSGVPDRFSGSGSGTDFTLTISSLQAEDVAVYYCWQGTH
FPGTFGGGTKVEIK.

Therefore, in some embodiments, the anti-EGFRviii V_{H} domain comprises the amino acid sequence

(SEQ ID NO: 101)
EIQLVQSGAEVKKPGESLRISCKGSGFNIEDYYIHWVRQMPGKGLEWMG
RIDPENDETKYGPIFQGHVTISADTSINTVYLQWSSLKASDTAMYYCAF
RGGVYWGQGTTVTVSS;

and the anti-EGFRviii V_{L} domain comprises the amino acid sequence

(SEQ ID NO: 102)
DVVMTQSPLSLPVTLGQPASISCKSSQSLLDSDGKTYLNWLQQRPGQSP
RRLISLVSKLDSGVPDRFSGSGSGTDFTLKISRVEAEDVGVYYCWQGTH
FPGTFGGGTKVEIK.

Therefore, in some embodiments, the anti-EGFRviii V_{H} domain comprises the amino acid sequence

(SEQ ID NO: 103)
EIQLVQSGAEVKKPGATVKISCKGSGFNIEDYYIHWVQQAPGKGLEWMG
RIDPENDETKYGPIFQGRVTITADTSTNTVYMELSSLRSEDTAVYYCAF
RGGVYWGQGTTVTVSS;

and the anti-EGFRviii V_{L} domain comprises the amino acid sequence

(SEQ ID NO: 104)
DVVMTQSPLSLPVTLGQPASISCKSSQSLLDSDGKTYLNWLQQRPGQSP
RRLISLVSKLDSGVPDRFSGSGSGTDFTLKISRVEAEDVGVYYCWQGTH
FPGTFGGGTKVEIK

Therefore, in some embodiments, the anti-EGFRviii V_{H} domain comprises the amino acid sequence

(SEQ ID NO:105)
EIQLQQSGAELVKPGASVKLSCTGSGFNIEDYYIHWVKQRTEQGLEWIG
RIDPENDETKYGPIFQGRATITADTSSNTVYLQLSSLTSEDTAVYYCAF
RGGVYWGPGTTLTVSS;

and the anti-EGFRviii V_{L} domain comprises the amino acid sequence

(SEQ ID NO: 106)
HMDVVMTQSPLTLSVAIGQSASISCKSSQSLLDSDGKTYLNWLLQRPGQ
SPKRLISLVSKLDSGVPDRFTGSGSGTDFTLRISRVEAEDLGIYYCWQG
THFPGTFGGGTKLEIK.

The heavy and light chains are preferably separated by a linker. Suitable linkers for scFv antibodies are known in the art. In some embodiments, the linker comprises the amino acid sequence GGGGSGGGGGGGGS (SEQ ID NO: 17). The scFv can have the formula NH₃-V_{H}-linker-V_{L}-COOH or NH₃-V_{L}-linker-V_{H}-COOH.

In some embodiments, the anti-IL13rα2 scFv comprises an amino acid sequence:

(SEQ ID NO: 23)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSGGGGSGGGGSGGGGSDIQMTQSPSSLSASV
GDRVTITCRASQDVSTAVAWYQQKPGKAPKLLIYSASFLYSGVPSRFSG
SGSGTDFTLTISSLQPEDFATYYCQQYLYHPATFGQGTKVEIKRTVAAP
S

In some embodiments, the anti-IL13rα2 scFv comprises an amino acid sequence:

(SEQ ID NO: 24)
DIQMTQSPSSLSASVGDRVTITCRASQDVSTAVAWYQQKPGKAPKLLIY
SASFLYSGVPSRFSGSGSGTDFTLTISSLQPEDFATYYCQQYLYHPATF
GQGTKVEIKRTVAAPSGGGGSGGGGSGGGGSEVQLVESGGGLVQPGGSL
RLSCAASGFTFSDSWIHWVRQAPGKGLEWVAWISPYGGSTYYADSVKGR
FTISADTSKNTAYLQMNSLRAEDTAVYYCARRHWPGGFDYWGQGTLVTV
S.

In some embodiments, the anti-EGFRviii scFv comprises an amino acid sequence:

(SEQ ID NO: 57)
DIQMTQSPSSLSASVGDRVTITCRASQRASESVDNYGISFMNAWYQQKP
GKAPKAASRQGSGSGVPSRFSGSGSGTDFTLTISSLQPEDFATYYCQQS
KEVPWTGTKVEIKRTVAAPSGGGGSGGGGSGGGGSEVQLVESGGGLVQP
GGSLRLSCAASGFTFSNYLMNWVRQAPGKGLEWVRIDPYDGDIDYNQNF
KDGGSTYYADSVKGRFTISADTSKNTAYLQMNSLRAEDTAVYYCAGYGT
AYGVDYYWGQGTLVTVS.

In some embodiments, the anti-EGFRviii scFv comprises an amino acid sequence:

(SEQ ID NO: 58)
EVQLVESGGGLVQPGGSLRLSCAASGFTFSNYLMNWVRQAPGKGLEWVR
IDPYDGDIDYNQNFKDGGSTYYADSVKGRFTISADTSKNTAYLQMNSLR
AEDTAVYYCAGYGTAYGVDYYWGQGTLVTVSGGGGSGGGGSGGGGSDIQ
MTQSPSSLSASVGDRVTITCRASQRASESVDNYGISFMNAWYQQKPGKA
PKAASRQGSGSGVPSRFSGSGSGTDFTLTISSLQPEDFATYYCQQSKEV
PWTGTKVEIKRTVAAPS.

In some embodiments, the anti-EGFRviii scFv comprises an amino acid sequence:

(SEQ ID NO: 59)
EIQLVQSGAEVKKPGATVKISCKGSGFNIEDYYIHWVQQAPGKGLEWMG
RIDPENDETKYGPIFQGRVTITADTSTNTVYMELSSLRSEDTAVYYCAF
RGGVYWGQGTTVTVSSGGGGSGGGGSGGGGSGGGGSDVVMTQSPDSLAV
SLGERATINCKSSQSLLDSDGKTYLNWLQQKPGQPPKRLISLVSKLDSG
VPDRFSGSGSGTDFTLTISSLQAEDVAVYYCWQGTHFPGTFGGGTKVEI
K.

Therefore, in some embodiments, the antigen binding domain has the amino acid sequence:

(SEQ ID NO: 18, EGFRviii.tandem.IL13Rα2)
EVQLVESGGGLVQPGGSLRLSCAASGFTFSDSWIHWVRQAPGKGLEWVA
WISPYGGSTYYADSVKGRFTISADTSKNTAYLQMNSLRAEDTAVYYCAR
RHWPGGFDYWGQGTLVTVSGGGGSGGGGSGGGGSDIQMTQSPSSLSASV
GDRVTITCRASQDVSTAVAWYQQKPGKAPKLLIYSASFLYSGVPSRFSG
SGSGTDFTLTISSLQPEDFATYYCQQYLYHPATFGQGTKVEIKRTVAAP
SGGGGSGGGGSGGGGSDIQMTQSPSSLSASVGDRVTITCRASQRASESV
DNYGISFMNAWYQQKPGKAPKAASRQGSGSGVPSRFSGSGSGTDFTLTI
SSLQPEDFATYYCQQSKEVPWTGTKVEIKRTVAAPSGGGGSGGGGSGGG
GSEVQLVESGGGLVQPGGSLRLSCAASGFTFSNYLMNWVRQAPGKGLEW
VRIDPYDGDIDYNQNFKDGGSTYYADSVKGRFTISADTSKNTAYLQMNS
LRAEDTAVYYCAGYGTAYGVDYYWGQGTLVTVS.

Therefore, in some embodiments, the antigen binding domain has the amino acid sequence:

(SEQ ID NO: 25
DIQMTQSPSSLSASVGDRVTITCRASQRASESVDNYGISFMNAWYQQKP
GKAPKAASRQGSGSGVPSRFSGSGSGTDFTLTISSLQPEDFATYYCQQS
KEVPWTGTKVEIKRTVAAPSGGGGSGGGGSGGGGSEVQLVESGGGLVQP
GGSLRLSCAASGFTFSNYLMNWVRQAPGKGLEWVRIDPYDGDIDYNQNF
KDGGSTYYADSVKGRFTISADTSKNTAYLQMNSLRAEDTAVYYCAGYGT
AYGVDYYWGQGTLVTVSGGGGSGGGGSGGGGSEVQLVESGGGLVQPGGS
LRLSCAASGFTFSDSWIHWVRQAPGKGLEWVAWISPYGGSTYYADSVKG
RFTISADTSKNTAYLQMNSLRAEDTAVYYCARRHWPGGFDYWGQGTLVT
VSGGGGSGGGGSGGGGSDIQMTQSPSSLSASVGDRVTITCRASQDVSTA
VAWYQQKPGKAPKLLIYSASFLYSGVPSRFSGSGSGTDFTLTISSLQPE
DFATYYCQQYLYHPATFGQGTKVEIKRTVAAPS,

IL13Rα2.tandem.EGFRviii.tandem).

Therefore, in some embodiments, the antigen binding domain has the amino acid sequence:

(SEQ ID NO: 60, EGFRviii-IL13Rα2)
EIQLVQSGAEVKKPGATVKISCKGSGFNIEDYYIHWVQQAPGKGLEWMG
RIDPENDETKYGPIFQGRVTITADTSTNTVYMELSSLRSEDTAVYYCAF
RGGVYWGQGTTVTVSSGGGGSGGGGSGGGGSGGGGSDVVMTQSPDSLAV
SLGERATINCKSSQSLLDSDGKTYLNWLQQKPGQPPKRLISLVSKLDSG
VPDRFSGSGSGTDFTLTISSLQAEDVAVYYCWQGTHFPGTFGGGTKVEI
KGGGGSGGGGSGGGGSDIQMTQSPSSLSASVGDRVTITCRASQRASESV
DNYGISFMNAWYQQKPGKAPKAASRQGSGSGVPSRFSGSGSGTDFTLTI
SSLQPEDFATYYCQQSKEVPWTGTKVEIKRTVAAPSGGGGSGGGGSGGG
GSEVQLVESGGGLVQPGGSLRLSCAASGFTFSNYLMNWVRQAPGKGLEW
VRIDPYDGDIDYNQNFKDGGSTYYADSVKGRFTISADTSKNTAYLQMNS
LRAEDTAVYYCAGYGTAYGVDYYWGQGTLVTVS.

Also disclosed is humanized antibody or antibody fragment comprising the CDRs disclosed herein. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and coworkers (Jones et al, Nature, 321:522-525 (1986); Riechmann et al, Nature, 332:323-327 (1988); Verhoeyen et al, Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592, 106; EP 519,596; Padlan, 1991, Molecular Immunology, 28 (4/5): 489-498; Studnicka et al, Protein Engineering, 7 (6): 805-814 (1994); and Roguska et al, PNAS, 91:969-973 (1994)) or chain shuffling (U.S. Pat. No. 5,565,332), the contents of which are incorporated herein by reference herein in their entirety.

Additional CAR constructs are described, for example, in Fresnak A D, et al. Engineered T cells: the promise and challenges of cancer immunotherapy. Nat Rev Cancer. 2016 Aug. 23; 16 (9): 566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or SCAR.

TRUCKs (T cells redirected for universal cytokine killing) co-express a chimeric antigen receptor (CAR) and an antitumor cytokine. Cytokine expression may be constitutive or induced by T cell activation. Targeted by CAR specificity, localized production of pro-inflammatory cytokines recruits endogenous immune cells to tumor sites and may potentiate an antitumor response.

Universal, allogeneic CAR T cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds to a tumor ligand, thereby enhancing tumor homing.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

Marked CAR T cells express a CAR plus a tumor epitope to which an existing monoclonal antibody agent binds. In the setting of intolerable adverse effects, administration of the monoclonal antibody clears the CAR T cells and alleviates symptoms with no additional off-tumor effects.

A tandem CAR (TanCAR) T cell expresses a single CAR consisting of two linked single-chain variable fragments (scFvs) that have different affinities fused to intracellular co-stimulatory domain(s) and a CD33 domain. TanCAR T cell activation is achieved only when target cells co-express both targets.

A dual CAR T cell expresses two separate CARs with different ligand binding targets; one CAR includes only the CD33 domain and the other CAR includes only the co-stimulatory domain(s). Dual CAR T cell activation requires co-expression of both targets on the tumor.

A safety CAR (sCAR) consists of an extracellular scFv fused to an intracellular inhibitory domain. SCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIγ, FcγRIIγ, FcεRIβ (FCERIB), and FcεRIγ (FCERIG). Variant CD3ζ signaling domains lacking one or more ITAM domains may also find use in the herein described CARs. Such variant CD3ζ signaling domains include those described by Bridgeman et al., Clin. Exp. Immunol. 175:258-267 (2013), which is herein incorporated by reference.

In some embodiments, the costimulatory signaling region comprises the amino acid sequence

(SEQ ID NO: 19, CD28.41BB)
RSKRSRLLHSDYMNMTPRRPGPTRKHYQPYAPPRDFAAYRSKRGRKKLL
YIFKQPFMRPVQTTQEEDGCSCRFPEEEEGGCEL.

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

In some embodiments, the CD3ζ region comprises the amino acid sequence

(SEQ ID NO: 20)
RVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR.

First-generation CARs typically had the intracellular domain from the CD33 chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. Preclinical studies have indicated that the second generation of CAR designs improves the antitumor activity of T cells. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD33 signaling domain by itself or combined with any other desired cytoplasmic domain(s) useful in the context of the CAR of the invention. For example, the cytoplasmic domain of the CAR can comprise a CD37 chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4 (2): 89-99 (2004)). The hinge sequence may be positioned between the antigen recognition moiety (e.g., anti-CD19, -CD20, or -CD22 scFv) and the transmembrane domain. The hinge sequence can be any suitable sequence derived or obtained from any suitable molecule. In some embodiments, for example, the hinge sequence is derived from a CD8a molecule or a CD28 molecule.

In some embodiments, the transmembrane domain is a CD8 hinge domain comprising the amino acid sequence

(SEQ ID NO: 21)
TTTPAPRPPTPAPTIASQPLSLRPEACRPAAGGAVHTRGLDFACDIYIW
APLAGTCGVLLLSLVIT.

In some embodiments, the transmembrane domain is a CD28 transmembrane domain comprising the amino acid sequence

(SEQ ID NO: 26)
IEVMYPPPYLDNEKSNGTIIHVKGKHLCPSPLFPGPSKPFWVLVVVGGV
LACYSLLVTVAFIIFWV.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In some embodiments, the anti-BCA binding agent is single chain variable fragment (scFv) antibody. The affinity/specificity of an anti-BCA scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the anti-BCA binding agent is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Also disclosed are bi-specific CARs that target a BCA and at least one additional tumor antigen. Also disclosed are CARs designed to work only in conjunction with another CAR that binds a different antigen, such as a tumor antigen. For example, in these embodiments, the endodomain of the disclosed CAR can contain only an signaling domain (SD) or a co-stimulatory signaling region (CSR), but not both. The second CAR (or endogenous T-cell) provides the missing signal if it is activated. For example, if the disclosed CAR contains an SD but not a CSR, then the immune effector cell containing this CAR is only activated if another CAR (or T-cell) containing a CSR binds its respective antigen. Likewise, if the disclosed CAR contains a CSR but not a SD, then the immune effector cell containing this CAR is only activated if another CAR (or T-cell) containing an SD binds its respective antigen.

Tumor antigens are proteins that are produced by tumor cells that elicit an immune response, particularly T-cell mediated immune responses. The additional antigen binding domain can be an antibody or a natural ligand of the tumor antigen. The selection of the additional antigen binding domain will depend on the particular type of cancer to be treated. Tumor antigens are well known in the art and include, for example, a glioma-associated antigen, carcinoembryonic antigen (CEA), EGFRvIII, IL-IIRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, TIM3, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6, E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, TIM3, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvIII, IL-13Rα, CD123, CD19, TIM3, BCMA, GD2, CLL-1, CA-IX, MUCI, HER2, and any combination thereof.

Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG1 6, TA-90\Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

### Nucleic Acids and Vectors

Also disclosed are polynucleotides and polynucleotide vectors encoding the disclosed CARs that allow expression of the CARs in the disclosed immune effector cells. Nucleic acid sequences encoding the disclosed CARs, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Expression of nucleic acids encoding CARs is typically achieved by operably linking a nucleic acid encoding the CAR polypeptide to a promoter, and incorporating the construct into an expression vector. Typical cloning vectors contain transcription and translation terminators, initiation sequences, and promoters useful for regulation of the expression of the desired nucleic acid sequence.

The disclosed nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some embodiments, the polynucleotide vectors are lentiviral or retroviral vectors.

For example, disclosed herein is an expression vector, comprising a nucleic acid sequence encoding a CD40L polypeptide and a nucleic acid sequence encoding a bi-specific chimeric antigen receptor (CAR) polypeptide that selectively binds IL13rα2 and EGFRviii, operably linked to an expression control sequence.

In some embodiments, the nucleic acid sequence encoding a CD40L polypeptide and the nucleic acid sequence encoding a CAR polypeptide are linked by a nucleic acid sequence encoding a self-cleaving peptide. In some embodiments, the self-cleaving peptide comprises a 2A peptide (P2A).

In some embodiments, the expression vector has the formula:

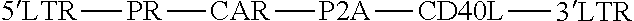

wherein “PR” represents the expression control sequence,
wherein “CAR” represents the nucleic acid sequence encoding the bi-specific CAR polypeptide,
wherein “P2A” represents the nucleic acid sequence encoding the self-cleaving peptide,
wherein “CD40L” represents the nucleic acid sequence encoding the CD40L polypeptide, and
wherein “-” represents a hydrogen bond or nucleic acid linker.

In some embodiments, the expression vector further comprises a nucleic acid sequence encoding a fluorescent tag prior to the 3′LTR.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, MND (myeloproliferative sarcoma virus) promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The promoter can alternatively be an inducible promoter. Examples of inducible promoters include, but are not limited to a metallothionine promoter, a glucocorticoid promoter, a progesterone promoter, and a tetracycline promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.).

### Immune Effector Cells

Also disclosed are immune effector cells that are engineered to express the disclosed CARs (also referred to herein as “CAR-T cells”). These cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dentritic cells, mast cells, neutrophils, basophils, eosinophils, or any combinations thereof. For example, the immune effector cells can comprise T lymphocytes, preferably cytotoxic T lymphocytes (CTLs).

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (Tc cells, or CTLs) destroy virally infected cells and tumor cells, and are also implicated in transplant rejection. These cells are also known as CD8+ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described-naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T-cell receptor (TCR) having one γ chain and one δ chain instead of α and β chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can also eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects. Although NK cells have a well-known role as killers of cancer cells, and NK cell impairment has been extensively documented as crucial for progression of MM (Godfrey J, et al. Leuk Lymphoma 2012 53:1666-1676; Fauriat C, et al. Leukemia 2006 20:732-733), the means by which one might enhance NK cell-mediated anti-MM activity has been largely unexplored prior to the disclosed CARs.

Epstein-Barr virus (EBV)-induced lymphoproliferative diseases (EBV-LPDs) and other EBV-associated cancers are a significant cause of morbidity and mortality for recipients of allogeneic hematopoietic cell transplantation (HCT) or solid organ transplants (SOT), particularly in those who have received certain T-cell reactive Abs to prevent or treat GVHD. Prophylaxis and treatment by the adoptive transfer of autologous or allogeneic EBV-specific cytotoxic T cells and the subsequent long-term restoration of immunity against EBV-associated lymphoproliferation have provided positive outcomes in the management of these uniformly fatal complications of allogeneic tissue transfer. Therefore, in some embodiments, the disclosed immune effector cells that comprise one or more of the CAR polypeptides of the present invention are allogeneic or autologous EBV-specific cytotoxic T lymphocytes (CTLs). For example, generation of EBV-specific cytotoxic T cells may involve isolating PBMCs from of an EBV-seropositive autologous or allogenic donor and enriching them for T cells by depletion of monocytes and NK cells. EBV-specific cytotoxic T cells may also be produced by contacting donor PBMCs or purified donor T cells with a “stimulator” cell that expresses one or more EBV antigen(s) and presents the EBV antigen(s) to unstimulated T cells, thereby causing stimulation and expansion of EBV-specific CTLs. EBV antigens include, for example, latent membrane protein (LMP) and EBV nuclear antigen (EBNA) proteins, such as LMP-1, LMP-2A, and LMP-2B and EBNA-1, EBNA-2, EBNA-3A, EBNA-3B, EBNA-3C and EBNA-LP. Cytotoxic T cells that comprise T cell receptor(s) which recognize one or more EBV-specific antigens are deemed to have been “sensitized” to those EBV antigen(s) and are therefore termed “EBV-sensitized cytotoxic T cells” herein. Known methods for generating allogeneic or autologous EBV-specific cytotoxic T cell populations that may comprise one or more of the CAR polypeptides of the present invention are described, for example, in Barker et al., Blood 2010 116 (23): 5045-49; Doubrovina, et al., Blood 2012 119 (11): 2644-56; Koehne, et al. Blood 2002 99 (5): 1730-40; and Smith et al. Cancer Res 2012 72 (5): 1116-25, which are incorporated by reference for these teachings.

### Therapeutic Methods

Immune effector cells expressing the disclosed CARs can elicit an anti-tumor immune response against IL13rα2 and EGFRviii expressing cancer cells. IL13rα2 is expressed in ovarian cancer (Kioi, M., et al. Cancer, 107:1407-1418), Squamous Cell Carcinoma of the Head and Neck (Kawakami M, et al. Clin Cancer Res 2003; 9:6381-8), Glioblastoma (Zeng J, et al. Am J Transl Res. 2020 12 (8): 4702-4714 (, and liver.

The anti-tumor immune response elicited by the disclosed CAR-modified immune effector cells may be an active or a passive immune response. In addition, the CAR-mediated immune response may be part of an adoptive immunotherapy approach in which CAR-modified immune effector cells induce an immune response specific to IL13rα2 and/or EGFRviii.

Adoptive transfer of immune effector cells expressing chimeric antigen receptors is a promising anti-cancer therapeutic. Following the collection of a patient's immune effector cells, the cells may be genetically engineered to express the disclosed CAR polypeptide and CD40L, then infused back into the patient. Moreover, immune effector cells obtained from a donor other than the patient (i.e., allogeneic to the patient) may be genetically engineered to express the disclosed CAR polypeptide and CD40L, then the CAR-containing cells infused into the patient. In one specific embodiment, the immune effector cells are allogeneic EBV-specific cytotoxic T cells.

The disclosed CAR-modified immune effector cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat MM. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed CAR-modified immune effector cells are administered to a patient in conjunction with (e.g., before, simultaneously or following) any number of relevant treatment modalities, including but not limited to thalidomide, dexamethasone, bortezomib, and lenalidomide. In further embodiments, the CAR-modified immune effector cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The cancer of the disclosed methods can be any IL13rα2 and EGFRviii expressing cell in a subject undergoing unregulated growth, invasion, or metastasis. Cancers that express IL13rα2 and EGFRviii include Glioblastoma, prostate, ovarian, and squamous cell carcinoma of the head and neck (Kioi, M., et al., Cancer, 2006. 107 (6): 1407-1418; Kawakami, M., et al., Clinical Cancer Research, 2003. 9 (17): 6381-6388; Zeng, J., et al., Am J Transl Res, 2020. 12 (8): 4702-4714; Olapade-Olaopa, E. O., et al., Br J Cancer, 2000. 82 (1): 186-94; Wikstrand, C. J., et al., Cancer Res, 1997. 57 (18): 4130-40).

In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed CARs can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed CARs can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHlgM12B7), CTLA-4 (Ipilimumab (MDX-010), Tremelimumab (CP-675,206)), IDO, B7-H3 (MGA271), B7-H4, TIM3, LAG-3 (BMS-986016). Techniques for combining CARs with checkpoint inhibitors in immune effector cells and use thereof for the treatment of various disorders are described, for example, in WO 2017/040945, which is incorporated by reference herein.

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MEDI4736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed CARs can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec STI571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with a CARs for treating the disorders as described above may be an anti-cancer cytokine, chemokine, or combination thereof. Examples of suitable cytokines and growth factors include IFNy, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-la from the human CXC and C—C chemokine families. Suitable cytokines include cytokine derivatives, cytokine variants, cytokine fragments, and cytokine fusion proteins.

In some embodiments, a therapeutic agent for use in combination with a CARs for treating the disorders as described above may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent for use in combination with a CARs for treating the disorders as described above may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use in combination with an CARs for treating the disorders as described above may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed CARs is administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed CARs is administered in combination with surgery.

CAR-T cells may be designed in several ways that enhance tumor cytotoxicity and specificity, evade tumor immunosuppression, avoid host rejection, and prolong their therapeutic half-life. TRUCK (T-cells Redirected for Universal Cytokine Killing) T cells for example, possess a CAR but are also engineered to release cytokines such as IL-12 that promote tumor killing. Because these cells are designed to release a molecular payload upon activation of the CAR once localized to the tumor environment, these CAR-T cells are sometimes also referred to as ‘armored CARs’. Several cytokines as cancer therapies are being investigated both pre-clinically and clinically, and may also prove useful when similarly incorporated into a TRUCK form of CAR-T therapy. Among these include IL-2, IL-3. IL-4, IL-5, IL-6, IL-7, IL-10, IL-12, IL-13, IL-15, IL-18, M-CSF, GM-CSF, IFN-α, IFN-γ, TNF-α, TRAIL, FLT3 ligand, Lymphotactin, and TGF-β (Dranoff 2004). “Self-driving” or “homing” CAR-T cells are engineered to express a chemokine receptor in addition to their CAR. As certain chemokines can be upregulated in tumors, incorporation of a chemokine receptor aids in tumor trafficking to and infiltration by the adoptive T-cell, thereby enhancing both specificity and functionality of the CAR-T (Moon 2011). Universal CAR-T cells also possess a CAR, but are engineered such that they do not express endogenous TCR (T-cell receptor) or MHC (major histocompatibility complex) proteins. Removal of these two proteins from the signaling repertoire of the adoptive T-cell therapy prevents graft-versus-host-disease and rejection, respectively. Armored CAR-T cells are additionally so named for their ability to evade tumor immunosuppression and tumor-induced CAR-T hypofunction. These particular CAR-Ts possess a CAR, and may be engineered to not express checkpoint inhibitors. Alternatively, these CAR-Ts can be co-administered with a monoclonal antibody (mAb) that blocks checkpoint signaling. Administration of an anti-PDL1 antibody significantly restored the killing ability of CAR TILs (tumor infiltrating lymphocytes). While PD1-PDL1 and CTLA-4-CD80/CD86 signaling pathways have been investigated, it is possible to target other immune checkpoint signaling molecules in the design of an armored CAR-T including LAG-3, Tim-3, IDO-1, 2B4, and KIR. Other intracellular inhibitors of TILs include phosphatases (SHP1), ubiquitin-ligases (i.e., cbl-b), and kinases (i.e., diacylglycerol kinase). Armored CAR-Ts may also be engineered to express proteins or receptors that protect them against or make them resistant to the effects of tumor-secreted cytokines. For example, CTLs (cytotoxic T lymphocytes) transduced with the double negative form of the TGF-β receptor are resistant to the immunosuppression by lymphoma secreted TGF-β. These transduced cells showed notably increased antitumor activity in vivo when compared to their control counterparts.

Tandem and dual CAR-T cells are unique in that they possess two distinct antigen binding domains. A tandem CAR contains two sequential antigen binding domains facing the extracellular environment connected to the intracellular costimulatory and stimulatory domains. A dual CAR is engineered such that one extracellular antigen binding domain is connected to the intracellular costimulatory domain and a second, distinct extracellular antigen binding domain is connected to the intracellular stimulatory domain. Because the stimulatory and costimulatory domains are split between two separate antigen binding domains, dual CARs are also referred to as “split CARs”. In both tandem and dual CAR designs, binding of both antigen binding domains is necessary to allow signaling of the CAR circuit in the T-cell. Because these two CAR designs have binding affinities for different, distinct antigens, they are also referred to as “bi-specific” CARs.

One primary concern with CAR-T cells as a form of “living therapeutic” is their manipulability in vivo and their potential immune-stimulating side effects. To better control CAR-T therapy and prevent against unwanted side effects, a variety of features have been engineered including off-switches, safety mechanisms, and conditional control mechanisms. Both self-destruct and marked/tagged CAR-T cells for example, are engineered to have an “off-switch” that promotes clearance of the CAR-expressing T-cell. A self-destruct CAR-T contains a CAR, but is also engineered to express a pro-apoptotic suicide gene or “elimination gene” inducible upon administration of an exogenous molecule. A variety of suicide genes may be employed for this purpose, including HSV-TK (herpes simplex virus thymidine kinase), Fas, iCasp9 (inducible caspase 9), CD20, MYC TAG, and truncated EGFR (endothelial growth factor receptor). HSK for example, will convert the prodrug ganciclovir (GCV) into GCV-triphosphate that incorporates itself into replicating DNA, ultimately leading to cell death. iCasp9 is a chimeric protein containing components of FK506-binding protein that binds the small molecule AP1903, leading to caspase 9 dimerization and apoptosis. A marked/tagged CAR-T cell however, is one that possesses a CAR but also is engineered to express a selection marker. Administration of a mAb against this selection marker will promote clearance of the CAR-T cell. Truncated EGFR is one such targetable antigen by the anti-EGFR mAb, and administration of cetuximab works to promotes elimination of the CAR-T cell. CARs created to have these features are also referred to as sCARs for ‘switchable CARs’, and RCARs for ‘regulatable CARs’. A “safety CAR”, also known as an “inhibitory CAR” (iCAR), is engineered to express two antigen binding domains. One of these extracellular domains is directed against a tumor related antigen and bound to an intracellular costimulatory and stimulatory domain. The second extracellular antigen binding domain however is specific for normal tissue and bound to an intracellular checkpoint domain such as CTLA4, PD1, or CD45. Incorporation of multiple intracellular inhibitory domains to the iCAR is also possible. Some inhibitory molecules that may provide these inhibitory domains include B7-H1, B7-1, CD160, PIH, 2B4, CEACAM (CEACAM-1. CEACAM-3, and/or CEACAM-5), LAG-3, TIGIT, BTLA, LAIR1, and TGFβ-R. In the presence of normal tissue, stimulation of this second antigen binding domain will work to inhibit the CAR. It should be noted that due to this dual antigen specificity, iCARs are also a form of bi-specific CAR-T cells. The safety CAR-T engineering enhances specificity of the CAR-T cell for tumor tissue, and is advantageous in situations where certain normal tissues may express very low levels of a tumor associated antigen that would lead to off target effects with a standard CAR (Morgan 2010). A conditional CAR-T cell expresses an extracellular antigen binding domain connected to an intracellular costimulatory domain and a separate, intracellular costimulator. The costimulatory and stimulatory domain sequences are engineered in such a way that upon administration of an exogenous molecule the resultant proteins will come together intracellularly to complete the CAR circuit. In this way, CAR-T activation can be modulated, and possibly even ‘fine-tuned’ or personalized to a specific patient. Similar to a dual CAR design, the stimulatory and costimulatory domains are physically separated when inactive in the conditional CAR; for this reason these too are also referred to as a “split CAR”.

In some embodiments, two or more of these engineered features may be combined to create an enhanced, multifunctional CAR-T. For example, it is possible to create a CAR-T cell with either dual- or conditional-CAR design that also releases cytokines like a TRUCK. In some embodiments, a dual-conditional CAR-T cell could be made such that it expresses two CARs with two separate antigen binding domains against two distinct cancer antigens, each bound to their respective costimulatory domains. The costimulatory domain would only become functional with the stimulatory domain after the activating molecule is administered. For this CAR-T cell to be effective the cancer must express both cancer antigens and the activating molecule must be administered to the patient; this design thereby incorporating features of both dual and conditional CAR-T cells.

Typically, CAR-T cells are created using α-β T cells, however γ-δ T cells may also be used. In some embodiments, the described CAR constructs, domains, and engineered features used to generate CAR-T cells could similarly be employed in the generation of other types of CAR-expressing immune cells including NK (natural killer) cells, B cells, mast cells, myeloid-derived phagocytes, and NKT cells. Alternatively, a CAR-expressing cell may be created to have properties of both T-cell and NK cells. In an additional embodiment, the transduced with CARs may be autologous or allogeneic.

Several different methods for CAR expression may be used including retroviral transduction (including γ-retroviral), lentiviral transduction, transposon/transposases (Sleeping Beauty and PiggyBac systems), and messenger RNA transfer-mediated gene expression. Gene editing (gene insertion or gene deletion/disruption) has become of increasing importance with respect to the possibility for engineering CAR-T cells as well. CRISPR-Cas9, ZFN (zinc finger nuclease), and TALEN (transcription activator like effector nuclease) systems are three potential methods through which CAR-T cells may be generated.

A number of embodiments of the invention have been described. Nevertheless, it will be understood that various modifications may be made without departing from the spirit and scope of the invention. Accordingly, other embodiments are within the scope of the following claims.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of providing an anti-cancer immunity in a subject with a cancer expressing IL13rα2 and EGFRviii, the method comprising administering to the subject an effective amount of the bi-specific chimeric antigen receptor (CAR) T cell of claim 25, thereby providing an anti-tumor immunity in the subject.

2-19. (canceled)

20. The method of claim 1, further comprising administering to the subject a checkpoint inhibitor.

21. The method of claim 20, wherein the checkpoint inhibitor comprises an anti-PD-1 antibody, anti-PD-L1 antibody, anti-CTLA-4 antibody, or a combination thereof.

22. The method of claim 1, wherein CAR T cells administered to the mammal are autologous to the subject.

23. The method of claim 1, wherein CAR T cells administered to the mammal are allogeneic to the subject.

24. The method of claim 1, further comprising suspending the CAR T cells in a solution comprising a Toll-like Receptor (TLR) agonist.

25. A bi-specific chimeric antigen receptor (CAR) T cell, comprising an immune effector cell engineered to express (a) a CD40 agonist and (b) a chimeric antigen receptor polypeptide that selectively binds IL13rα2 and EGFRviii.

26. The bi-specific CAR T cell of claim 25, wherein the chimeric antigen receptor polypeptide that selectively binds IL13rα2 and EGFRviii comprises an antigen-binding domain, a transmembrane domain, an intracellular signaling domain, and a co-stimulatory signaling region, wherein the antigen binding domain comprises an IL13rα2 binding domain and an EGFRviii binding domain.

27. The bi-specific CAR T cell of claim 25, wherein the chimeric antigen receptor polypeptide is defined by the formula:
IL13rα2V_{L}-EGFRviiiV_{H}-EGFRviiiV_{L}-IL13rα2V_{H}-TM-CSR/ISD;
EGFRviiiV_{L}-IL13rα2V_{H}-IL13rα2V_{L}-EGFRviiiV_{H}-TM-CSR/ISD;
IL13rα2V_{H}-EGFRviiiV_{L}-EGFRviiiV_{H}-IL13rα2V_{L}-TM-CSR/ISD;
EGFRviiiV_{H}-IL13rα2V_{L}-IL13rα2V_{H}-EGFRviiiV_{L}-TM-CSR/ISD;
IL13rα2V_{H}-IL13rα2V_{L}-EGFRviiiV_{L}-EGFRviiiV_{H}-TM-CSR/ISD;
EGFRviiiV_{H}-EGFRviiiV_{L}-IL13rα2V_{L}-IL13rα2V_{H}-TM-CSR/ISD;
IL13rα2V_{L}-IL13rα2V_{H}-EGFRviiiV_{H}-EGFRviiiV_{L}-TM-CSR/ISD; or
EGFRviiiV_{L}-EGFRviiiV_{H}-IL13rα2V_{H}-IL13rα2V_{L}-TM-CSR/ISD;
wherein “IL13rα2V_{H}” represents an anti-IL13rα2 V_{H} domain,
wherein “IL13rα2V_{L}” represents an anti-IL13rα2 V_{L} domain,
wherein “EGFRviiiV_{H}” represents an anti-EGFRviii V_{H} domain,
wherein “EGFRviiiV_{L}” represents an anti-EGFRviii V_{L} domain,
wherein “TM” represents a transmembrane domain,
wherein “CSR/ISD” represents a co-stimulatory signaling region and/or intracellular signaling domain, and
wherein “-” represents a peptide bond or linker.

28. The bi-specific CAR T cell of claim 26, wherein the IL13rα2 binding domain is a single-chain variable fragment (scFv) of an antibody that specifically binds IL13rα2 comprising a variable heavy (V_{H}) domain having CDR1, CDR2 and CDR3 sequences and a variable light (V_{L}) domain having CDR1, CDR2 and CDR3 sequences, wherein the CDR1 sequence of the V_{H} domain comprises the amino acid sequence NYLMN (SEQ ID NO:1); the CDR2 sequence of the V_{H} domain comprises the amino acid sequence RIDPYDGDIDYNQNFKD (SEQ ID NO:2); the CDR3 sequence of the V_{H} domain comprises the amino acid sequence GYGTAYGVDY (SEQ ID NO:3); the CDR1 sequence of the V_{L} comprises the amino acid sequence RASESVDNYGISFMN (SEQ ID NO:4); the CDR2 sequence of the V_{L} domain comprises the amino acid sequence AASRQGSG (SEQ ID NO:5); and the CDR3 sequence of the V_{L} domain comprises the amino acid sequence QQSKEVPWT (SEQ ID NO:6).

29. The bi-specific CAR T cell of claim 28, wherein the anti-IL13rα2 scFv V_{H} domain comprises the amino acid sequence SEQ ID NO:7.

30. The bi-specific CAR T cell of claim 28, wherein the anti-IL13rα2 scFv V_{L} domain comprises the amino acid sequence SEQ ID NO:8.

31. The bi-specific CAR T cell of claim 26, wherein the EGFRviii binding domain is a single-chain variable fragment (scFv) of an antibody that specifically binds EGFRviii comprising a variable heavy (V_{H}) domain having CDR1, CDR2 and CDR3 sequences and a variable light (V_{L}) domain having CDR1, CDR2 and CDR3 sequences, wherein the CDR1 sequence of the V_{H} domain comprises the amino acid sequence DSWIH (SEQ ID NO:9); the CDR2 sequence of the V_{H} domain comprises the amino acid sequence AWISPY (SEQ ID NO:10); the CDR3 sequence of the V_{H} domain comprises the amino acid sequence RRHWPGGFD (SEQ ID NO: 11); the CDR1 sequence of the V_{L} comprises the amino acid sequence DVSTAV (SEQ ID NO: 12); the CDR2 sequence of the V_{L} domain comprises the amino acid sequence LLIYSASFLY (SEQ ID NO: 13); and the CDR3 sequence of the V_{L} domain comprises the amino acid sequence QQYLYHPATFGQ (SEQ ID NO:14).

32. The bi-specific CAR T cell of claim 31, wherein the anti-EGFRviii scFv V_{H} domain comprises the amino acid sequence SEQ ID NO:55.

33. The bi-specific CAR T cell of claim 31, wherein the anti-EGFRviii scFv V_{L} domain comprises the amino acid sequence SEQ ID NO:56.

34. The bi-specific CAR T cell of claim 26, wherein the linker comprises the amino acid sequence SEQ ID NO:17.

35. The bi-specific CAR T cell of claim 26, wherein the antigen binding domain comprises the amino acid sequence SEQ ID NO: 60.

36. The bi-specific CAR T cell of claim 26, wherein the costimulatory signaling region comprises the cytoplasmic domain of a costimulatory domain selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

37. The bi-specific CAR T cell of claim 36, wherein costimulatory signaling region comprises the amino acid sequence SEQ ID NO:19.

38. The bi-specific CAR T cell of claim 26, wherein the intracellular signaling domain comprises a CD3 zeta (CD3ζ) signaling domain.

39. The bi-specific CAR T cell of claim 38, wherein the CD3 zeta region comprises the amino acid sequence SEQ ID NO:20.

40. The bi-specific CAR T cell of claim 26, wherein transmembrane domain is a CD8 linker and hinge domain comprising the amino acid sequence SEQ ID NO:21.

41. The bi-specific CAR T cell of claim 26, wherein transmembrane domain is a CD28 linker and transmembrane domain comprising the amino acid sequence SEQ ID NO:26.

42. The bi-specific CAR T cell of claim 25, wherein the CD40 agonist is a CD40L polypeptide comprising the amino acid sequence SEQ ID NO:22.

43. The bi-specific CAR T cell of claim 25, wherein the immune effector cell is selected from the group consisting of an αβT cell, γδT cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

44. The bi-specific CAR T cell of claim 25, wherein the CAR T cells are suspended in a solution comprising a Toll-like Receptor (TLR) agonist

45. An expression vector, comprising a nucleic acid sequence encoding a CD40L polypeptide and a nucleic acid sequence encoding a bi-specific chimeric antigen receptor (CAR) polypeptide that selectively binds IL13rα2 and EGFRviii, operably linked to an expression control sequence.

46-49. (canceled)
